(19) United States
(12) Patent Application Publication
(10) Pub. No.: US 2026/0297025 A1
(43) Pub. Date: Oct. 1, 2026

(54) PINCER-TYPE DIPHENYL SULFIDE COMPOUND, AND PREPARATION METHOD AND USE THEREOF

(71) Applicant: Hunan Institute of Science and Technology, Yueyang City (CN)

(72) Inventors: Kewen TANG, Yueyang City (CN); Hongwei LONG, Yueyang City (CN); Ming YI, Yueyang City (CN); Wanru WANG, Yueyang City (CN)

(73) Assignee: Hunan Institute of Science and Technology, Yueyang City (CN)

(21) Appl. No.: 19/277,118

(22) Filed: Jul. 22, 2025

(30) Foreign Application Priority Data
Mar. 28, 2025 (CN) 202510387006.7

(51) Int. Cl. C07C 321/30; C07C 319/20; C07C 323/20; C22B 3/34; C22B 11/00

(52) U.S. Cl. CPC C07C 321/30; C07C 319/20; C07C 323/20; C22B 3/34; C22B 11/046; C22B 11/048

(57) ABSTRACT

A structurally symmetrical diphenyl sulfide compound, and a preparation method and use thereof are provided. The structurally symmetrical (pincer-type) diphenyl sulfide compound (E_{S}/E_{O}) is used as an exemplified extractant. Principal active sites of pincer-type extractants are positioned on both flanks in a framework of the diphenyl sulfide compound, enabling formation of dual coordination architectures with the Au(III) and the Pd(II) in precious metals (PMs), while a central sulfur atom exhibits extraction capability specific to Pd.

### CROSS REFERENCE TO RELATED APPLICATION

This patent application claims the benefit and priority of Chinese Patent Application No. 2025103870067 filed with the China National Intellectual Property Administration on Mar. 28, 2025, the disclosure of which is incorporated by reference herein in its entirety as part of the present application.

### TECHNICAL FIELD

The present disclosure belongs to the technical field of resource recovery, and specifically relates to a pincer-type diphenyl sulfide compound, and a preparation method and use thereof.

### BACKGROUND

Precious metals (PMs), such as gold (Au) and palladium (Pd), possess exceptional physical and chemical properties including superior ductility, electrical conductivity, and outstanding catalytic performance. In recent years, their applications in the electronics and catalytic industries have witnessed significant growth. Against the backdrop of this surge, there is an urgent need to recover the PMs from secondary resources such as electronic wastes and spent catalysts to meet the escalating demand for non-renewable PM resources.

Generally, hydrometallurgical processes (e.g., extraction, precipitation, adsorption, and ion exchange) have been employed for PM recovery from the secondary resources, which involves precise recovery of PM ions from leachates. Among these methods, liquid-liquid extraction demonstrates advantages of high capacity, low cost, and operational simplicity, holding promise for large-scale applications. The strong coordination between functional atoms of extractants and the PM ions in different states is the main reason for the successful extraction of the PMs from an aqueous phase by the extractants. Building on this foundation, the development and synthesis of innovative extractants focus on incorporating multiple active sites and optimizing molecular architectures. For instance, atoms with strong interactions towards Au(III) and Pd(II), specifically nitrogen-based elements (such as N and P) and oxygen-based elements (such as O and S), are engineered into extractants for the PM recovery. Compared to nitrogen-based extractants that suffer from toxicity and solubility limitations, oxygen-based extractants exhibit greater potential for effective extraction of Au(III) and Pd(II), particularly benefiting from their superior affinity for the Au(III) and the Pd(II) that facilitates preferential coordination with target PM ions during the extraction. This is represented by thioether-based extractants, where “S” atoms function as “soft acids” according to the Hard and Soft Acids and Bases (HSAB) theory, specifically recognizing the “soft base” characteristics of Au(III) and Pd(II).

However, due to complex hydrochemical conditions of leachates (such as extremely low PM concentrations and coexistence of multiple metal ions), the selectivity of extractants designed solely based on functional atoms of active sites toward coexisting metal ions is unsatisfactory.

### SUMMARY

In view of this, the present disclosure is intended to provide a structurally symmetrical diphenyl sulfide compound, and a preparation method and use thereof. The structurally symmetrical diphenyl sulfide compound provided by the present disclosure could be used as an extractant to selectively recover Au(III) and Pd(II) from secondary resources with a high extraction efficiency.

The present disclosure provides a structurally symmetrical diphenyl sulfide compound having a structure selected from the group consisting of Formula I (denoted as E_{S}) and Formula II (denoted as E_{O}):

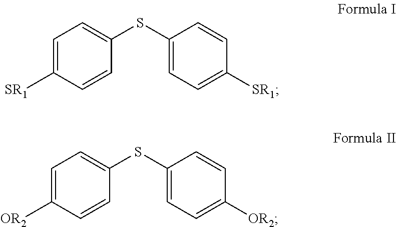

where

R₁ in the Formula I is a C4-C8 alkyl group; and R₂ in the Formula II is a C4-C8 alkyl group.

The present disclosure provides the structurally symmetrical (pincer-type) diphenyl sulfide compound (E_{S}/E_{O}) being used as an extractant, adopting a paired complexation strategy (i.e., two extractant molecules cooperatively sandwich two metal ions for co-extraction), enabling effective and selective recovery of Au(III) and Pd(II) from secondary resources. Principal active sites of pincer-type extractants are positioned on both flanks in a structure of the diphenyl sulfide compound, enabling formation of dual coordination architectures with the Au(III) and the Pd(II) in PMs, while a central S atom exhibits extraction capability specific to Pd. Specifically, S atoms on both sides of the E_{S} exhibit extraction capability specific to both Au and the Pd, whereas the central S atom exhibits extraction capability specific to the Pd. For PM extraction by the E_{O}, the Au is coordinated through O atoms on both sides of the E_{O}, while a central S atom exhibits extraction capability specific to the Pd, thus enabling effective and selective recovery of the Au(III) and the Pd(II) from the secondary resources.

Furthermore, the present disclosure enhances extractant hydrophobicity by extending an alkyl chain length on both sides of the extractant, thereby reducing the dissolution loss of the extractant in acidic leachates and improving operational stability for better reusability. For the E_{O}, longer alkyl chains increase the electronegativity of oxygen atoms, further improving extraction activity; conversely, for the E_{S}, shorter alkyl chains minimize steric hindrance and preserve sulfur atoms functionality, thereby improving extraction activity. Additionally, the incorporation of benzene rings into a thioether structure eliminates pungent odors.

The present disclosure prepares diphenyl sulfide compounds with optimized active sites and structures by modulating functional atoms (S or O) and carbon chain lengths (C4-C8 alkyl groups) at terminal positions. The E_{S} exhibits an extraction capacity of 818.15 mg/g for Au and an extraction capacity of 522.12 mg/g for Pd. Experimental results demonstrate that the diphenyl sulfide compounds provided by the present disclosure achieve near-quantitative recovery (approximately 100%) of Au and Pd from real-world electronic wastes and spent catalysts, while base metal ions exhibit minimal extraction efficiency (less than 1.7%). Furthermore, the extractants retain 90.7% to 94.5% extraction efficiency for the Au and the Pd after 10 reuse cycles.

### BRIEF DESCRIPTION OF THE DRAWINGS

To describe the technical solutions in embodiments of the present disclosure or in the prior art more clearly, drawings required in the embodiments are briefly described below. Apparently, the drawings in the following description show merely some embodiments of the present disclosure, and other drawings could still be derived from these drawings by those of ordinary skill in the art without creative efforts.

FIG. 1 shows a synthetic route of the diphenyl sulfide extractant according to an embodiment of the present disclosure;

FIG. 2A shows a ¹H Nuclear Magnetic Resonance (NMR) spectrum of the E_{S}−1;

FIG. 2B shows a ¹³C NMR spectrum of the E_{S}−1;

FIG. 3A shows a ¹H NMR spectrum of the E_{S}−2;

FIG. 3B shows a ¹³C NMR spectrum of the E_{S}−2;

FIG. 4A shows a ¹H NMR spectrum of the E_{S}−3;

FIG. 4B shows a ¹³C NMR spectrum of the E_{S}−3;

FIG. 5A shows a ¹H NMR spectrum of the E_{O}−1;

FIG. 5B shows a ¹³C NMR spectrum of the E_{O}−1;

FIG. 6A shows a ¹H NMR spectrum of the E_{O}−2;

FIG. 6B shows a ¹³C NMR spectrum of the E_{O}−2;

FIG. 7A shows a ¹H NMR spectrum of the E_{O}−3;

FIG. 7B shows a ¹³C NMR spectrum of the E_{O}−3;

FIG. 8A shows pseudo-first-order kinetic models for Au(III) and Pd(II) on the E_{S}−1;

FIG. 8B shows pseudo-first-order kinetic models for Au(III) and Pd(II) on the E_{O}−1;

FIG. 8C shows pseudo-second-order kinetic models for the Au(III) and the Pd(II) on the E_{S}−1;

FIG. 8D shows pseudo-second-order kinetic models for the Au(III) and the Pd(II) on the E_{O}−1;

FIG. 9A shows saturated extraction capacities (mol/mol) of the extractants for the Au(III);

FIG. 9B shows saturated extraction capacities (mol/mol) of the extractants for the Pd(II);

FIG. 9C shows saturated extraction capacities (mg/g) of the extractants for the Au(III);

FIG. 9D shows saturated extraction capacities (mg/g) of the extractants for the Pd(II);

FIG. 10A shows extraction of the Au(III) and the Pd(II) from simulated mixed metal ion solutions;

FIG. 10 shows extraction of the Au(III) and the Pd(II) from simulated mixed metal ion solutions;

FIG. 11A shows metal concentrations in a PCB leachate;

FIG. 11B shows metal concentrations in the PCB leachate;

FIG. 11C shows metal concentrations in a CPU leachate;

FIG. 11D shows metal concentrations in the CPU leachate;

FIG. 12 shows Fourier-transform infrared spectroscopy (FT-IR) spectra of the E_{S}−1 after stripping of the Au(III) and the Pd(II);

FIG. 13 shows a quantitative NMR spectrum for the solubility of E_{S}−1 in water;

FIG. 14 shows influence of diluents of the extractants on extraction efficiency of the Au(III) and the Pd(II);

FIG. 15A shows influence of a HCl concentration on extraction efficiency of the Au(III);

FIG. 15B shows influence of a HCl concentration on extraction efficiency of the Pd(II);

FIG. 16A shows influence of a concentration of the extractants on extraction efficiency of the Au(III);

FIG. 16B shows influence of a concentration of the extractants on extraction efficiency of the Pd(II);

FIG. 17A shows extraction kinetics of the Au(III) during extraction;

FIG. 17B shows extraction kinetics of the Pd(II) during extraction;

FIG. 18A shows maximum extraction capacities of E_{S} for the Au(III) and the Pd(II);

FIG. 18B shows maximum extraction capacities of E_{O} for the Au(III) and the Pd(II);

FIG. 19A shows FT-IR spectra of the extractant before and after coordination with the Au(III) and the Pd(II);

FIG. 19B shows X-ray Photoelectron Spectroscopy (XPS) deconvolution results of S·2p spectra for the E_{S}−1 and E_{S}−1−Pd;

FIG. 20A shows an electrostatic potential (ESP) map of the E_{S};

FIG. 20B shows an electrostatic potential (ESP) map of the E_{O};

FIG. 21A shows structure of E_{S}−16C;

FIG. 21B shows comparison of extraction efficiency between the E_{S}−1 and the E_{S}−16C;

FIG. 22 shows influences of H⁺ and Cl⁻ concentrations on the extraction performance of the Au(III) and the Pd(II);

FIG. 23 shows Job's plots for E_{S}−1−Au(III) and E_{S}−1−Pd(II) complexes;

FIG. 24 shows relationship between lg[D] and lg[Cl⁻];

FIG. 25 shows Ultraviolet-visible spectroscopy (UV-Vis) spectra of the E_{S} before and after the extraction of the Au(III);

FIG. 26 shows dual descriptor of the E_{S}−1;

FIG. 27 shows optimized structures of E_{S}−1−Au and E_{S}−1−Pd complexes;

FIG. 28 shows scanning electron microscopy (SEM) images of real-world PCB boards and their leachate solutions;

FIG. 29 shows SEM images of real-world CPUs and their leachate solutions;

FIG. 30 shows Au recovered using 2 mM E_{S}−1 under H⁺ concentrations of 0.1 M and 0.5 M;

FIG. 31A shows initial ion concentrations in a spent catalyst leachate dissolved by HNO₃;

FIG. 31B shows the extraction performance of the E_{S}−1 for the spent catalyst leachate;

FIG. 32A shows cyclic extraction-stripping tests for Au;

FIG. 32B shows cyclic extraction-stripping tests for Pd; and

FIG. 33 shows a schematic overview for the comprehensive performance of the E_{S}−1.

### DETAILED DESCRIPTION OF THE EMBODIMENTS

The present disclosure provides a structurally symmetrical diphenyl sulfide compound having a structure selected from the group consisting of Formula I and Formula II:

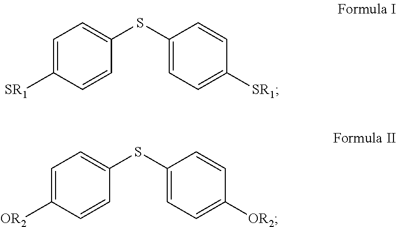

where
R₁ in the Formula I is a C4-C8 alkyl group; and R₂ in the Formula II is a C4-C8 alkyl group.

The present disclosure provides two diphenyl sulfide compounds with the Formula I and the Formula II structures, featuring “sulfur (S)” or “oxygen (O)” atoms on both sides, designated as E_{S} or E_{O}, respectively.

In the present disclosure, R₁ and R₂ each are independently selected from the group consisting of —C₄H₉, —C₆H₁₃, and —C₈H₁₇.

In the present disclosure, the diphenyl sulfide compound has a structure shown in any one of Formula 1 to Formula 6:

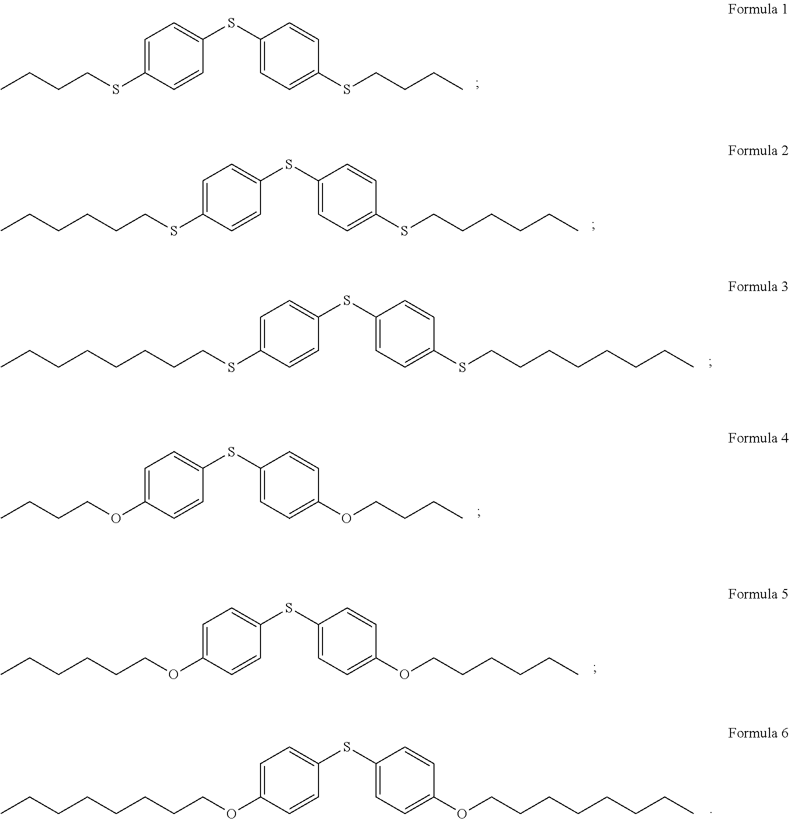

The present disclosure provides the structurally symmetrical (pincer-type) diphenyl sulfide compound (E_{S}/E_{O}) being used as an extractant, adopting a paired complexation strategy (i.e., two extractant molecules cooperatively sandwich two metal ions for co-extraction), enabling effective and selective recovery of Au(III) and Pd(II) from secondary resources. Principal active sites of the pincer-type extractants are positioned on both flanks in a framework of the diphenyl sulfide compound, enabling formation of dual coordination architectures with the Au(III) and the Pd(II) in PMs, while a central S atom exhibits extraction capability specific to Pd. Specifically, S atoms on both sides of the E_{S} exhibit extraction capability specific to both Au and the Pd, whereas the central S atom exhibits extraction capability specific to the Pd. For PM extraction by the E_{O}, the Au is coordinated through O atoms on both sides, while a central S atom exhibits extraction capability specific to the Pd, thus enabling effective and selective recovery of the Au(III) and the Pd(II) from the secondary resources.

Furthermore, the present disclosure enhances extractant hydrophobicity by extending a alkyl chain length on both sides of the extractant, thereby reducing dissolution loss of the extractant in acidic leachates and improving operational stability for better reusability. For the E_{O}, longer alkyl chains increase the electronegativity of oxygen atoms, further improving extraction activity; conversely, for the E_{S}, shorter alkyl chains minimize steric hindrance and preserve sulfur atoms functionality, thereby improving extraction activity. Additionally, incorporation of benzene rings into a thioether structure eliminates pungent odors.

The present disclosure further provides a method for preparing the diphenyl sulfide compound described in the above technical solutions, including the following steps:
mixing mercapto diphenyl sulfide or hydroxy diphenyl sulfide, a halogenated alkane, an organic solvent, an acid-binding agent, and an organic ammonium salt catalyst, and subjecting a resulting mixture to substitution to obtain the diphenyl sulfide compound,
where an alkane in the halogenated alkane is a C4-C8 alkane; the mercapto diphenyl sulfide includes 4,4′-dimercapto diphenyl sulfide; and the hydroxy diphenyl sulfide includes 4,4′-dihydroxy diphenyl sulfide.

In some embodiments of the present disclosure, the halogenated alkane includes a halogenated group selected from the group consisting of a halogenated butyl group, a halogenated hexyl group, and a halogenated octyl group. In some embodiments of the present disclosure, the organic solvent includes at least one selected from the group consisting of acetonitrile, acetone, and N,N-dimethylformamide (DMF). In some embodiments of the present disclosure, the acid-binding agent includes at least one selected from the group consisting of carbonates and sodium hydroxide, where the carbonates include at least one selected from the group consisting of potassium carbonate and cesium carbonate. In some embodiments of the present disclosure, the organic ammonium salt catalyst includes tetrabutylammonium bromide (TBAB).

In some embodiments of the present disclosure, the mercapto diphenyl sulfide has a structure shown in Formula III, and the hydroxy diphenyl sulfide has a structure shown in Formula IV;

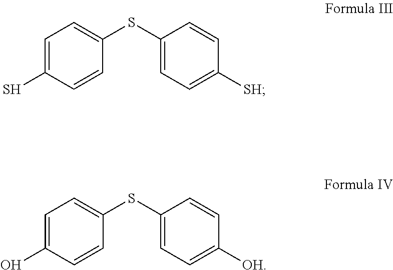 [SMILES: Sc1ccc(Sc2ccc(S)cc2)cc1 ; Oc1ccc(Sc2ccc(O)cc2)cc1]

In some embodiments of the present disclosure, a molar ratio of the mercapto diphenyl sulfide or the hydroxy diphenyl sulfide to the halogenated alkane is 1:2.2; a molar ratio of the mercapto diphenyl sulfide or the hydroxy diphenyl sulfide to the acid-binding agent is 1:2; a ratio of a molar amount of the mercapto diphenyl sulfide or the hydroxy diphenyl sulfide to a mass of the organic ammonium salt catalyst is 1 mmol:2 mg; a ratio of a molar amount of the mercapto diphenyl sulfide or the hydroxy diphenyl sulfide to a volume of the organic solvent is 10 mmol:15 mL. In some embodiments of the present disclosure, the substitution is conducted at a temperature of 70° C. to 90° C. (specifically 70° C., 75° C., 80° C., 85° C., or 90° C. in embodiments) for 24 hours to 60 hours (specifically 24 hours, 30 hours, 40 hours, 50 hours, 55 hours, or 60 hours in embodiments).

In some embodiments of the present disclosure, the method further includes after the substitution is completed, subjecting a resulting reaction solution to a post-treatment, where the post-treatment includes: subjecting the resulting reaction solution to solvent removal, and addition of an aqueous acid-binding agent, subjecting a resulting aqueous solution to extraction with an organic solvent (such as DCM), water washing with an organic phase, drying with anhydrous sodium sulfate, and then organic solvent removal to obtain the extractant.

The present disclosure provides use of the diphenyl sulfide compound described in the above technical solutions or the diphenyl sulfide compound prepared by the method described in the above technical solutions as an extractant in extraction of Au³⁺ and/or Pd²⁺.

In some embodiments of the present disclosure, the extraction includes: mixing an acidic leachate containing the Au³⁺ and/or the Pd²⁺ with an extractant dilution solution, and subjecting a resulting mixed solution to the extraction.

In some embodiments of the present disclosure, Au- and/or Pd-containing waste materials are subjected to mixed leaching with acidic reagents, thus yielding the acidic leachate containing the Au³⁺ and/or the Pd²⁺. In some embodiments of the present disclosure, the Au- and/or Pd-containing waste materials include one selected from the group consisting of PCB boards, CPU boards, and spent palladium catalysts.

In some embodiments of the present disclosure, a concentration of H⁺ in the acidic leachate ranges from 0.1 mol/L to 18 mol/L (specifically 0.1 mol/L, 0.2 mol/L, 0.3 mol/L, 0.4 mol/L, 0.5 mol/L, 1 mol/L, 5 mol/L, 10 mol/L, 15 mol/L, 16 mol/L, 17 mol/L, or 18 mol/L in embodiments), a concentration of the Au³⁺ in the acidic leachate ranges from 0 mol/L to 80 mg/L (specifically 0 mg/L, 2 mg/L, 5 mg/L, 10 mg/L, 20 mg/L, 30 mg/L, 40 mg/L, 50 mg/L, 60 mg/L, 70 mg/L, or 80 mg/L in embodiments), and a concentration of the Pd²⁺ in the acidic leachate ranges from 0 mol/L to 15 mg/L (specifically 0 mg/L, 3 mg/L, 4 mg/L, 5 mg/L, 6 mg/L, 8 mg/L, or 10 mg/L in embodiments). In some embodiments of the present disclosure, the concentration of the Au³⁺ and the concentration of Pd²⁺ are not 0 simultaneously.

In some embodiments of the present disclosure, a diluent in the extractant dilution solution includes at least one selected from the group consisting of DCM, DCE, toluene, n-hexane, petroleum ether, and kerosene. In some embodiments of the present disclosure, the extractant in the extractant dilution solution has a concentration of 1 mmol/L to 10 mmol/L (specifically 1 mmol/L, 2 mmol/L, 4 mmol/L, 5 mmol/L, 6 mmol/L, 8 mmol/L, or 10 mmol/L in embodiments). In some embodiments of the present disclosure, a volume ratio of the acidic leachate to the extractant dilution solution is in a range of (1-9):(1-9) (specifically 1:1, 1:2, 1:3, 1:4, 1:5, 1:6, 1:7, 1:8, 1:9, 3:4, 5:6, 9:1, 9:2, 9:3, 9:4, 9:5, 9:6, 9:7, or 9:8 in embodiments). In some embodiments of the present disclosure, the extraction is conducted for 4 hours to 15 hours (specifically 4 hours, 5 hours, 6 hours, 8 hours, 10 hours, 12 hours, 14 hours, or 15 hours in embodiments).

The present disclosure prepares diphenyl sulfide compounds with optimized active sites and structures by modulating functional atoms (S or O) and carbon chain lengths (C4-C8 alkyl groups) at terminal positions. The E_{S} exhibits an extraction capacity of 818.15 mg/g for Au and an extraction capacity of 522.12 mg/g for Pd. Experimental results demonstrate that the diphenyl sulfide compounds provided by the present disclosure achieve near-quantitative recovery (approximately 100%) of Au and Pd from real-world electronic waste and spent catalysts, while base metal ions exhibit minimal extraction efficiency (less than 1.7%). Furthermore, the extractants retain 90.7% to 94.5% extraction efficiency for the Au and the Pd after 10 reuse cycles.

In order to further illustrate the present disclosure, a structurally symmetrical diphenyl sulfide compound, and a preparation method and use thereof provided by the present disclosure will be described in detail below in conjunction with drawings and examples, but the these drawings and the examples should not be construed as limiting the scope of the present disclosure.

### Example 1

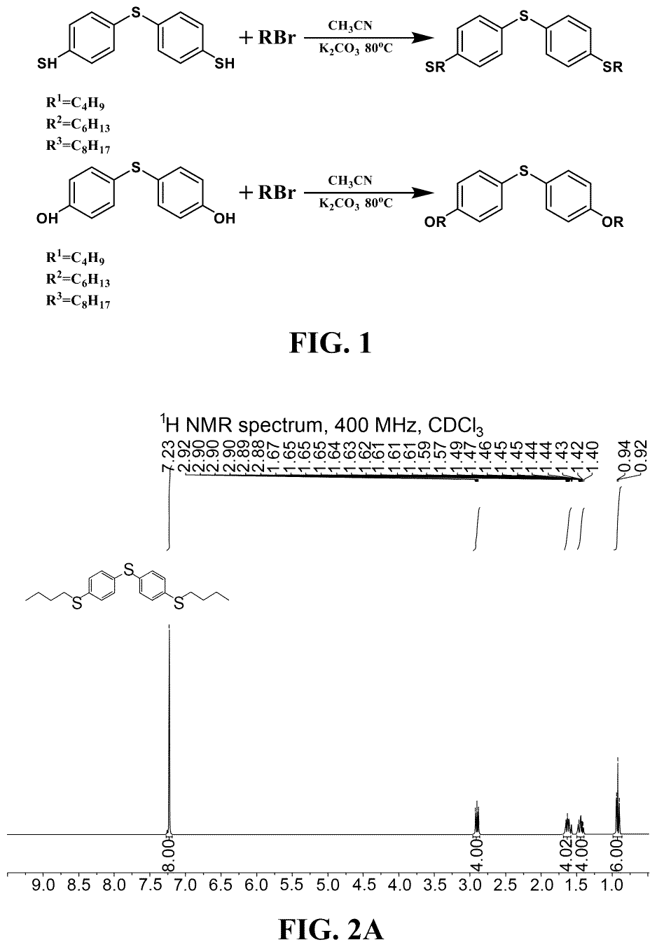

A synthesis procedure of E_{S}−1 was performed as follows: 4,4′-dimercapto diphenyl sulfide (2.5 g, 10 mmol), n-butyl bromide (22 mmol), acetonitrile (15 mL), potassium carbonate (K₂CO₃, 20 mmol, 2.76 g), and TBAB (20 mg) were mixed and stirred at 80° C. A resulting mixture was subjected to substitution. After 48 hours of the substitution, a solvent was removed by rotary evaporation. Water was then added to dissolve the K₂CO₃, and a reaction product was extracted with DCM. A resulting organic phase was washed three times with water, and dried over anhydrous sodium sulfate, and a solvent was removed by a rotary evaporator to obtain an extractant, designated as E_{S}−1. The compound E_{S}−1 appeared as a light green liquid, with the specific synthetic route illustrated in FIG. 1.

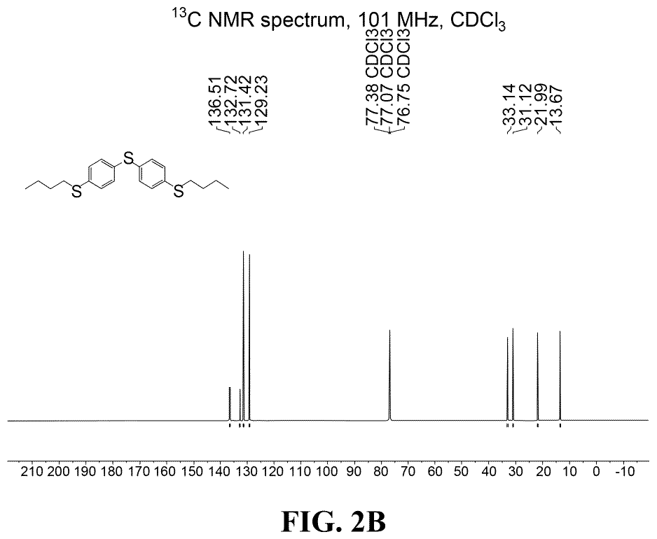

FIG. 2A shows a ¹H NMR spectrum of the E_{S}−1. FIG. 2B shows a ¹³C NMR spectrum of the E_{S}−1. Structural characterization data are: (¹H NMR (400 MHz, CDCl₃) δ 7.23 (s, 8H), 2.90 (dd, J=7.9, 6.8 Hz, 4H), 1.68-1.59 (m, 4H), 1.50-1.39 (m, 4H), 0.92 (t, J=7.3 Hz, 6H). ¹³C NMR (101 MHz, CDCl₃) δ 136.51, 132.72, 131.42, 129.23, 33.14, 31.12, 21.99, 13.67.

### Example 2

The synthesis of E_{S}−2 was the same as that of the E_{S}−1, except that 4,4′-dimercapto diphenyl sulfide (2.5 g, 10 mmol), hexyl bromide (22 mmol), acetonitrile (15 mL), potassium carbonate (K₂CO₃, 20 mmol, 2.76 g), and TBAB (20 mg) were mixed. A resulting extractant was designated as E_{S}−2. The compound E_{S}−2 appeared as a white solid, with the specific synthesis route illustrated in FIG. 1.

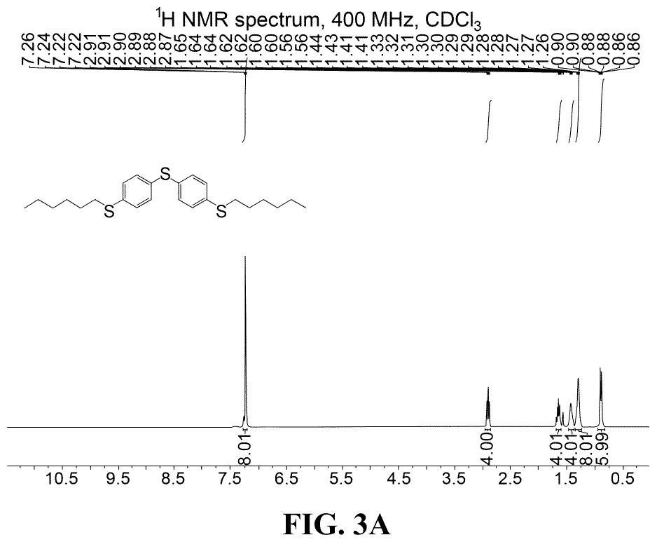

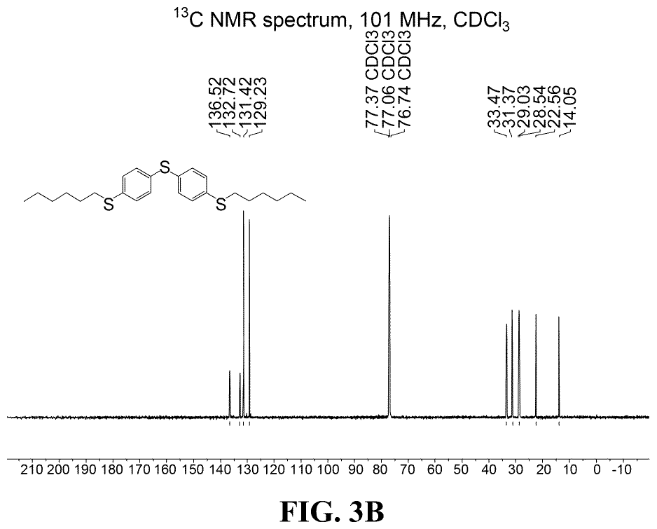

FIG. 3A shows a ¹H NMR spectrum of the E_{S}−2. FIG. 3B shows a ¹³C NMR spectrum of the E_{S}−2. Structural characterization data are: (¹H NMR (400 MHz, CDCl₃) δ 7.22 (d, J=1.7 Hz, 8H), 2.89 (td, J=7.4, 1.6 Hz, 4H), 1.68-1.59 (m, 4H), 1.46-1.37 (m, 4H), 1.34-1.23 (m, 8H), 0.88 (td, J=7.0, 1.7 Hz, 6H). ¹³C NMR (101 MHz, CDCl₃) δ 136.52, 132.72, 131.42, 129.23, 33.47, 31.37, 29.03, 28.54, 22.56, 14.05.

### Example 3

The synthesis of E_{S}−3 was the same as that of the E_{S}−1, except that 4,4′-dimercapto diphenyl sulfide (2.5 g, 10 mmol), octyl bromide (22 mmol), acetonitrile (15 mL), potassium carbonate (K₂CO₃, 20 mmol, 2.76 g) and TBAB (20 mg) were mixed. A resulting extractant was designated as E_{S}−3. The compound E_{S}−3 appeared as a white solid, with the specific synthesis route illustrated in FIG. 1.

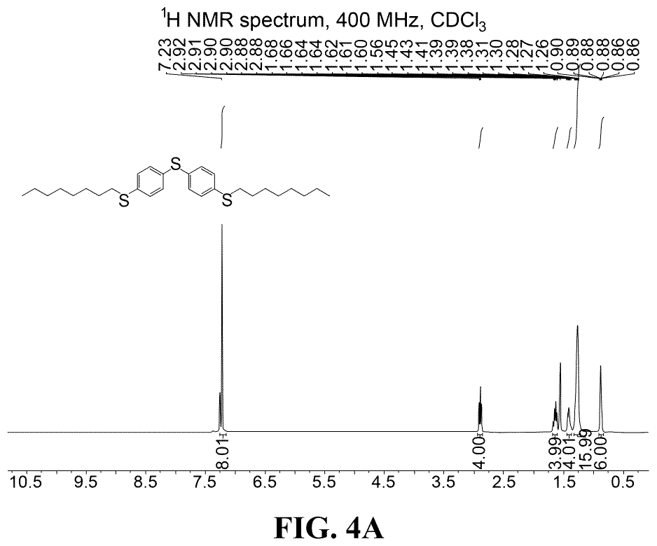

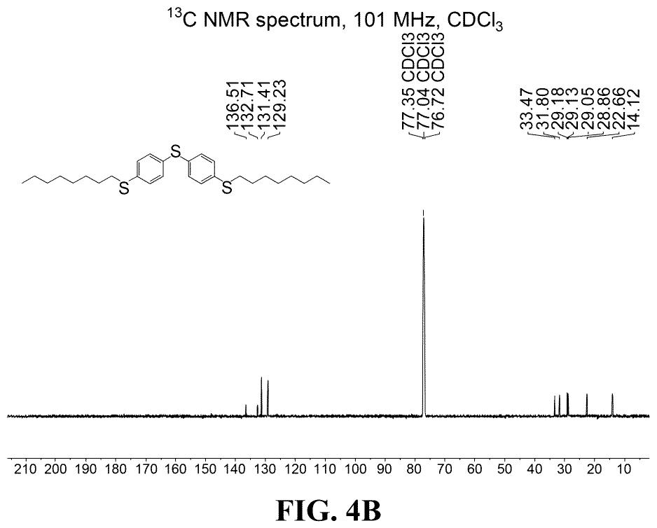

FIG. 4A shows a ¹H NMR spectrum of the E_{S}−3. FIG. 4B shows a ¹³C NMR spectrum of the E_{S}−3. Structural characterization data are: (¹H NMR (400 MHz, CDCl₃) δ 7.23 (s, 8H), 2.90 (td, J=7.4, 1.3 Hz, 4H), 1.69-1.60 (m, 4H), 1.41 (h, J=6.7 Hz, 4H), 1.28 (q, J=5.9 Hz, 16H), 0.92-0.84 (m, 6H). ¹³C NMR (101 MHz, CDCl₃) δ 136.51, 132.71, 131.41, 129.23, 33.47, 31.80, 29.18-28.86, 22.66, 14.12.

### Example 4

The synthesis of E_{O}−1 was the same as that of the E_{S}−1, except that 4,4′-dihydroxy diphenyl sulfide (2.18 g, 10 mmol), n-butyl bromide (22 mmol), acetonitrile (15 mL), potassium carbonate (K₂CO₃, 20 mmol, 2.76 g), and TBAB (20 mg) were mixed. A resulting extractant was designated as E_{O}−1. The compound E_{O}−1 appeared as a pale yellow solid, with the specific synthesis route illustrated in FIG. 1.

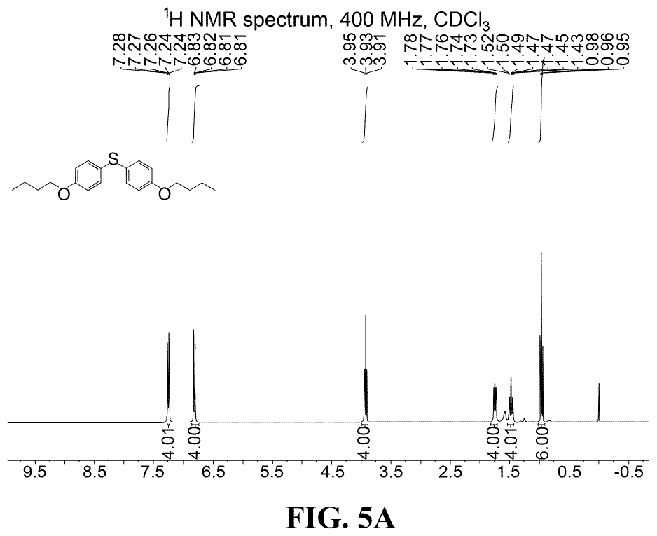

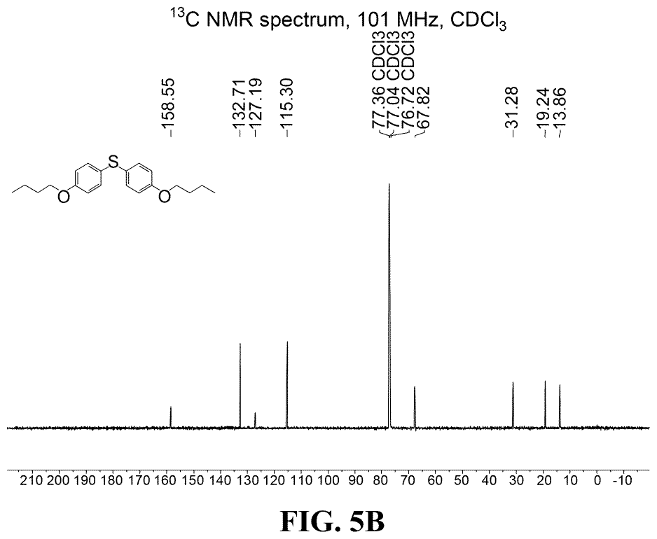

FIG. 5A shows a ¹H NMR spectrum of the E_{O}−1. FIG. 5B shows a ¹³C NMR spectrum of the E_{O}−1. Structural characterization data are: (¹H NMR (400 MHz, CDCl₃) δ 7.27-7.24 (m, 4H), 6.86-6.74 (m, 4H), 3.93 (t, J=6.5 Hz, 4H), 1.76 (dd, J=15.3, 6.1 Hz, 4H), 1.49 (dt, J=14.9, 7.4 Hz, 4H), 0.96 (t, J=7.4 Hz, 6H). ¹³C NMR (101 MHz, CDCl₃) δ 158.55, 132.71, 127.19, 115.30, 67.82, 31.28, 19.24, 13.86.

### Example 5

The synthesis of E_{O}−2 was the same as that of the E_{S}−1 except that 4,4′-dihydroxy diphenyl sulfide (2.18 g, 10 mmol), hexyl bromide (22 mmol), acetonitrile (15 mL), potassium carbonate (K₂CO₃, 20 mmol, 2.76 g), and TBAB (20 mg) were mixed. A resulting extractant was designated as E_{O}−2. The compound E_{O}−2 appeared as a white solid, with the specific synthesis route illustrated in FIG. 1.

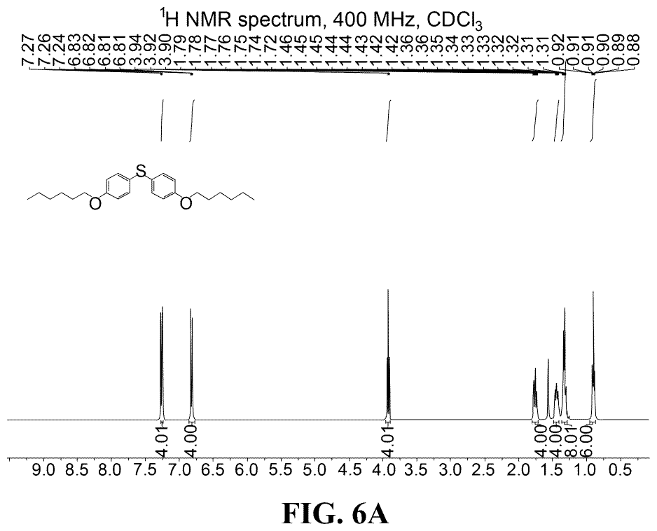

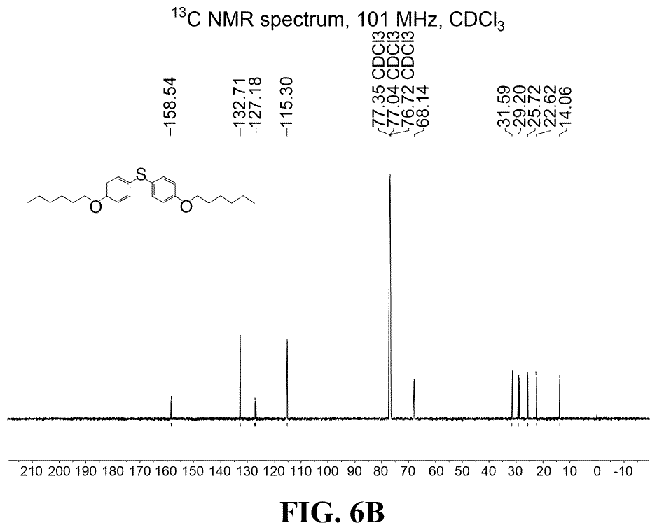

FIG. 6A shows a ¹H NMR spectrum of the E_{O}−2. FIG. 6B shows a ¹³C NMR spectrum of the E_{O}−2. Structural characterization data are: (¹H NMR (400 MHz, CDCl₃) δ 7.27-7.24 (m, 4H), 6.86-6.78 (m, 4H), 3.92 (t, J=6.6 Hz, 4H), 1.76 (p, J=6.7 Hz, 4H), 1.49-1.40 (m, 4H), 1.37-1.28 (m, 8H), 0.90 (td, J=5.7, 2.9 Hz, 6H). ¹³C NMR (101 MHz, CDCl₃) δ 158.54, 132.71, 127.18, 115.30, 68.14, 31.59, 29.20, 25.72, 22.62, 14.06.

### Example 6

The synthesis of E_{O}−3 was the same as that of the E_{S}−1 except that 4,4′-dihydroxy diphenyl sulfide (2.18 g, 10 mmol), octyl bromide (22 mmol), acetonitrile (15 mL), potassium carbonate (K₂CO₃, 20 mmol, 2.76 g), and TBAB (20 mg) were mixed. A resulting extractant was designated as E_{O}−3. The compound E_{O}−3 appeared as a white solid, with the specific synthesis route illustrated in FIG. 1.

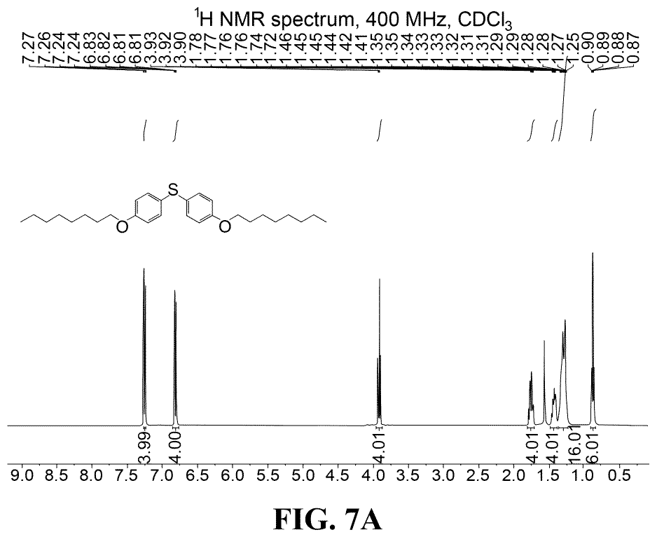

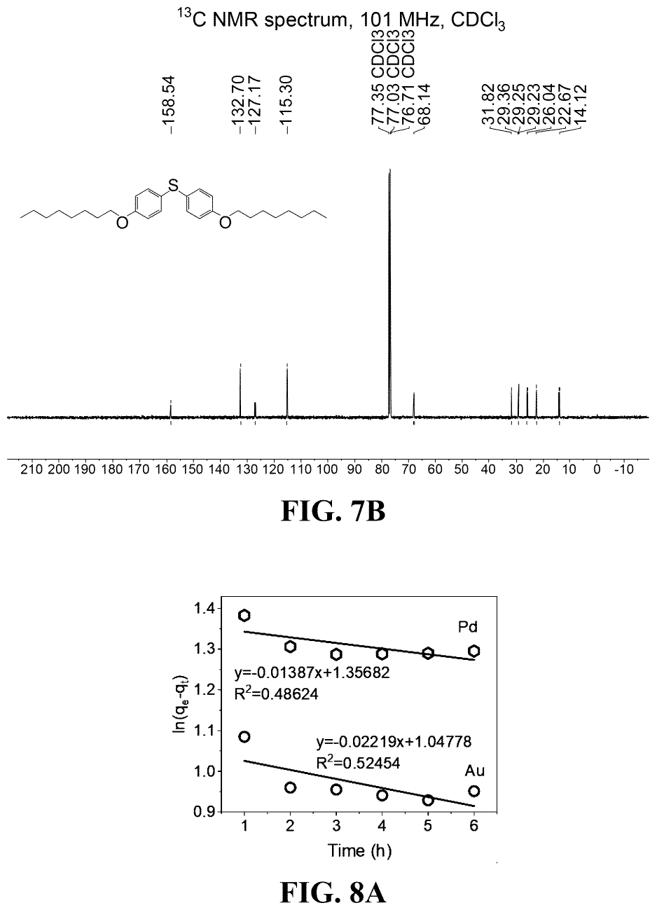

FIG. 7A shows a ¹H NMR spectrum of the E_{O}−3. FIG. 7B shows a ¹³C NMR spectrum of the E_{O}−3. Structural characterization data are: (¹H NMR (400 MHz, CDCl₃) δ 7.27-7.24 (m, 4H), 6.86-6.77 (m, 4H), 3.92 (t, J=6.6 Hz, 4H), 1.81-1.71 (m, 4H), 1.48-1.39 (m, 4H), 1.37-1.24 (m, 16H), 0.92-0.84 (m, 6H). ¹³C NMR (101 MHz, CDCl₃) δ 158.54, 132.70, 127.17, 115.30, 68.14, 31.82, 29.36, 29.25, 29.23, 26.04, 22.67, 14.12.

### Comparative Example 1

The synthesis of E_{S}−16C was the same as that of the E_{S}−1, except that octyl bromide was replaced by Br—(CH₂)₁₅—CH₃, and a resulting extractant was designated as E_{S}−16C.

Halogenated alkanes with carbon numbers less than C₄ show relatively low boiling points, while a synthesis temperature for preparing the extractant in the present disclosure is higher. Such halogenated alkanes with carbon numbers less than C₄ would volatilize during synthesis, resulting in that the preparation of the extractant could not be completed.

### Use Examples 1 to 3

A PCB (WeAct Studio, China) and a CPU (Intel, USA) were obtained from scrapped computers and circuit boards, while a spent catalyst was collected after use in hydrogenation reactions.

Real-world leaching feed solutions were prepared based on previous reports. Specifically, a 12*12 cm PCB board was soaked in 80 mL aqua regia for two days and filtered. A resulting extract was supplemented with 32 g solid NaOH, and a H⁺ concentration was adjusted to 0.1 M and 0.5 M using 1 mol/L NaOH aqueous solution.

A leaching process for CPU boards was identical to that for the PCB board, except that a single CPU board was soaked in 100 mL aqua regia for two days and filtered. An obtained extract was supplemented with 40.5 g NaOH, and a H⁺ concentration was adjusted to 0.1 M and 0.5 M using 5 mol/L NaOH solution.

Leaching of a spent palladium catalyst: the spent Pd catalyst (5 g) was washed three times with ethanol, thoroughly dried, dissolved in 100 mL HNO₃ (15.2 mol/L), and leached at 25° C. for 48 hours to obtain an acidic leachate. The acidic leachate contained Pd (13.92 ppm), Fe (4.11 ppm), and Cu (2.06 ppm).

Gold and palladium were separately extracted from 5 mL aliquots of the above leachates using 5 mL of 2 mM E_{S}−1 DCM solution with an extraction time of 4 hours.

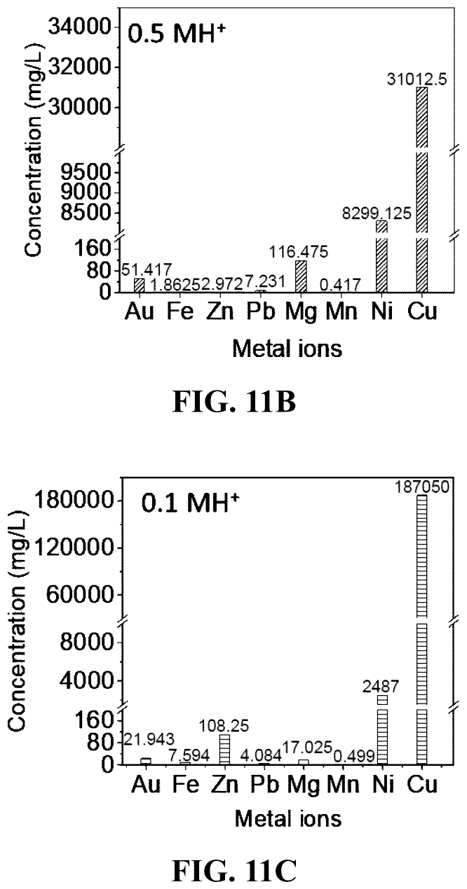

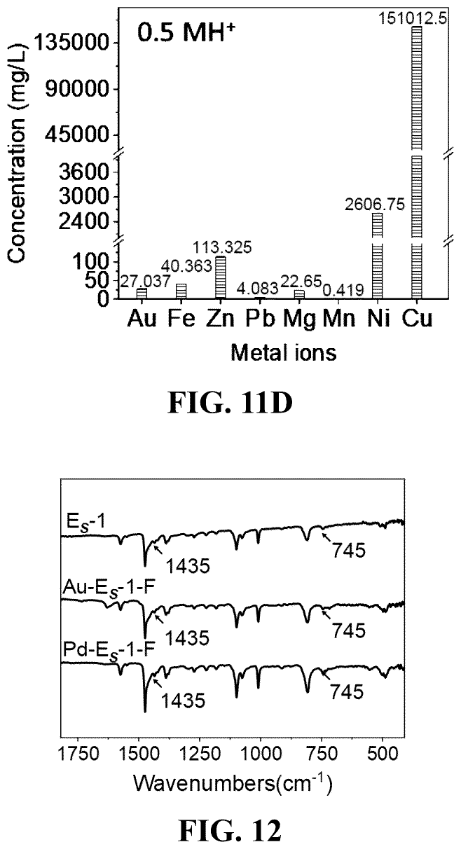

Subsequently, concentrations of metal ions in the leachates were determined by atomic absorption spectroscopy (AAS), ensuring that errors remained within acceptable limits. A metal composition of the original leachates is shown in FIGS. 11A-11D.

### Performance Testing

The present disclosure provides two diphenyl sulfide compounds, featuring “sulfur (S)” or “oxygen (O)” atoms on both sides, designated as E_{S} or E_{O}, respectively. During synthesis, 4,4′-dimercapto diphenyl sulfide and 4,4′-dihydroxy diphenyl sulfide were replaced by halogenated hydrocarbons with different carbon chain lengths (FIG. 1 to FIGS. 7A-7B). Extractants, with terminal carbon atoms numbering 4, 6, and 8, were designated as E_{S}−1, E_{S}−2, E_{S}−3 and E_{O}−1, E_{O}−2, E_{O}−3, respectively.

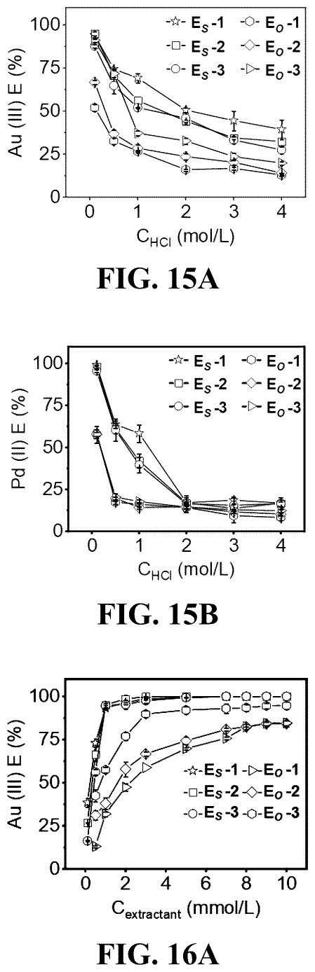

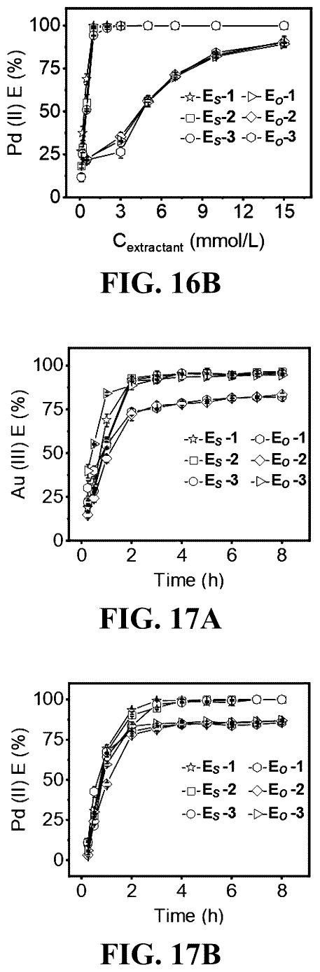

FIG. 14 shows influence of diluents of the extractant on extraction efficiency of Au(III) and Pd(II), with test conditions being [E_{S} and E_{O}]=5 mM, [Au(III)]=[Pd(II)]=1 mM, [HCl]=0.1 M, and a reaction time of 12 hours. FIG. 15A shows influence of a HCl concentration on extraction efficiency of the Au(III), FIG. 15B shows influence of a HCl concentration on extraction efficiency of the Pd(II), with test conditions of [E_{S}]=1 mM, [E_{O}]=5 mM, [Au(III)]=[Pd(II)]=1 mM, and a reaction time of 12 hours. FIG. 16A shows influence of a concentration of the extractants on extraction efficiency of the Au(III). FIG. 16B shows influence of a concentration of the extractants on extraction efficiency of the Pd(II). FIG. 17A shows extraction kinetics of the Au(III) during extraction. FIG. 17B shows extraction kinetics of the Pd(II) during extraction. Specific extraction procedures for FIG. 14 to FIG. 17B were performed as follows: first, the extractant was weighed and dissolved in DCM to prepare an organic phase (formulated according to the aforementioned test conditions); an aqueous phase consisted of 0.1 M HCl and 1 mM PM ions (Au(III) or Pd(II)); subsequently, a mixture of 5 mL each of the organic phase and the aqueous phase (O/A=1) was mechanically shaken at 230 rpm for a specified duration (i.e., the reaction time mentioned in the test conditions) at 25° C. After phase separation, a concentration of metal ions in the aqueous phase was determined using an atomic absorption spectroscopy (AAS). An extraction efficiency (E %) was calculated using the following formula:

$$E = \frac{{C}_{in} - {C}_{eq}}{{C}_{in}} \times 1 \; 00 \% ;$$

where

Cᵢₙ and C_{eq} (mM) refer to the concentration of metal ion in the aqueous phase before extraction and the concentration of the metal ion in the aqueous phase after extraction, respectively.

In a liquid-liquid extraction process, the coordination of AuCl₄⁻ or PdCl₄²⁻ in the aqueous phase with the extractants in the organic phase successfully achieved precise separation of PMs Au and Pd from different leachates. Consequently, a selection of the diluents significantly influenced extraction performance, as the polarity and viscosity of the diluents, along with the solubility of the extractants in solvents, directly affected extraction efficiency. For this purpose, six solvents including DCM, DCE, toluene, n-hexane, petroleum ether, and kerosene were selected as diluents for PM extraction in the present disclosure, while a concentration of the extractants was fixed at 5 mM.

It is evident from FIG. 14 that certain differences exist in extraction results among the various diluents. The first three diluents exhibit superior extraction performance compared to the latter three, which is likely attributable to differences in diluent polarity. The more polar diluents, the DCM, the DCE, and the toluene, provide satisfactory results for the extraction of the Au (99.3% to 100%) and the Pd (100%) using E_{S}, with both phases remaining transparent post-extraction. In contrast, the extraction efficiency for the Au in the n-hexane, the petroleum ether, and the kerosene were only 16.4% to 24.7%. Taking the n-hexane as an example, insoluble substances were observed in the organic phase after Pd extraction. These results indicate that the polarity of the solvent (diluent) played a critical role in the extraction, as it influences the solubility of complexes. Furthermore, the poor extraction efficiency of the E_{O} suggested that functional atoms also played a significant role in the extraction process. Given the optimal performance of the DCM among the six diluents tested, the DCM was selected as the diluent for subsequent experiments in the present disclosure.

As PMs in secondary resources are typically leached using strong acids, the present disclosure investigated the influence of the HCl concentration (ranging from 0.1 M to 4 M) on the extraction performance of the Au(III) and the Pd(II) with different extractants. FIGS. 15A-15B shows that as the HCl concentration in the aqueous phase increased, the performance of all studied extractants in PM extraction declines. This phenomenon is particularly pronounced in the extraction of the Pd, where the extraction performance of the E_{S} and the E_{O} sharply decreased to 16.3% and 14.4%, respectively, at 2 M HCl. These results indicate that high concentrations of H⁺ or Cl⁻ adversely affected coordination reactions between the extractants and metal ions. Studies reveal that the Au(III) exists as AuCl₄⁻ under low acidity, while coexistence of HAuCl₄⁻ at high acidity hinders binding of the AuCl₄⁻ with the extractants. Additionally, the increased Cl⁻ concentration in the aqueous phase negatively impacts the extraction, especially for the Pd(II). Since the Cl⁻ acts as a product in the coordination reactions, its abundance under high acidity promotes reverse reactions in the coordination reactions, as detailed below.

Furthermore, influence of varying carbon chain lengths in the diphenyl sulfide extractants on the extraction of the Au(III) and the Pd(II) differs significantly. Specifically, as demonstrated in FIG. 15A and FIG. 16A, the extractant E_{S}−1 with the shortest carbon chain exhibited the optimal extraction performance for the Au(III), while for the E_{O}, the extraction performance for the Au(III) followed the order E_{O}−3>E_{O}−2>E_{O}−1. This contrasting trend indicates that functional atoms play a decisive role in extraction of Au(III), particularly in the extractants with varying carbon chain lengths at their active sites. In contrast, the behavior for extraction of the Pd(II) differs markedly (FIG. 15B and FIG. 16B). The extraction efficiency of the E_{S} slightly declined with increasing terminal alkyl chain length, whereas the E_{O} maintained consistent performance across E_{O}−1, E_{O}−2, and E_{O}−3. These observations suggest that the functional atoms involved and the extraction mechanisms for the Au(III) and the Pd(II) are distinct.

Extraction kinetics have significant importance in PM extraction as they directly influence the overall process efficiency. Using 0.1 M HCl as an aqueous medium and DCM as the diluent, a extraction equilibrium time for 1 mM Au(III) and Pd(II) was examined, with E_{S} and E_{O} concentrations fixed at 2 mM and 10 mM, respectively. As shown in FIGS. 17A-17B, effective extraction of the Au(III) and the Pd(II) via the E_{S} is achieved within 2 hours to 3 hours, highlighting the efficiency of the selected extractants in removing the PM ions. During the same timeframe, E_{O} extraction also reached equilibrium, though its extraction efficiencies for the Au(III) and the Pd(II) remained at 77.0% to 92.0% and approximately 82.0%, respectively. To comprehensively interpret extraction kinetic mechanisms, data fitting analysis was conducted using a pseudo-first-order kinetic model (Equation 1) and a pseudo-second-order kinetic model (Equation 2) to evaluate the extraction process.

$$ln ( {q}_{e} - {q}_{t} ) = ln {q}_{e} - {k}_{1} t ; \qquad Equation 1$$

$$\frac{t}{{q}_{t}} = \frac{1}{{k}_{2} {q}_{e}^{2}} + \frac{1}{{q}_{e}} t ; \qquad Equation 2$$

where
qₑ and qₜ represent an extraction capacity at equilibrium and an extraction capacity at different time points, respectively. k₁ and k₂ refers to constants of a pseudo-first-order equation and a pseudo-second-order equation, respectively. t represents the extraction equilibrium time.

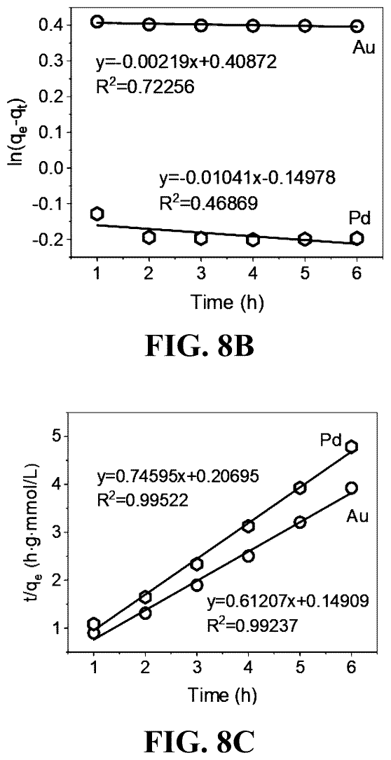

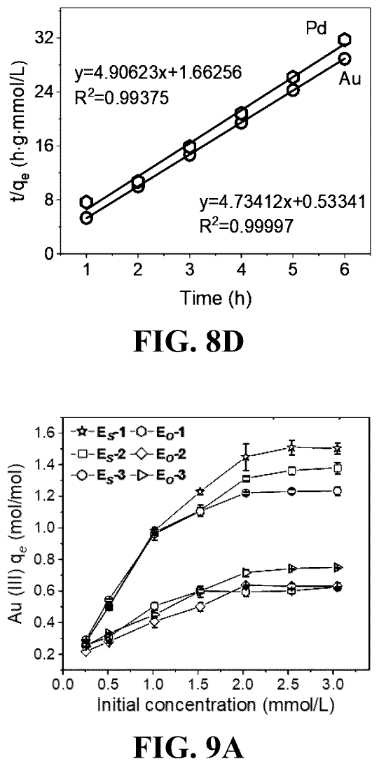

As shown in FIGS. 8A-8D (FIG. 8A showing the pseudo-first-order kinetic models for the Au(III) and the Pd(II) on the E_{S}−1; FIG. 8B showing the pseudo-first-order kinetic models for the Au(III) and the Pd(II) on the E_{O}−1; FIG. 8C showing the pseudo-second-order kinetic models for the Au(III) and Pd(II) on the E_{S}−1; and FIG. 8D showing the pseudo-second-order kinetic models for the Au(III) and Pd(II) on the E_{O}−1), extraction reactions of the E_{S}−1 and the E_{O}−3 are better described by the pseudo-second-order kinetic model, which suggested that chemical interactions between E_{S}/E_{O} and AuCl₄⁻/PdCl₄²⁻ serves as the rate-limiting step in the process.

### Validating Role of Functional Atoms in Revealing Structure-Selectivity Relationships Via Carbon Chain Length Modulation

The extraction capacity (qₑ) is critically important in industrial applications, serving as a key metric for evaluating extractant performance. In this context, a mass ratio (mg/g) accurately reflects both the efficiency and economic viability of the extractants. In the present disclosure, the extractant concentration was fixed at 1 mM, followed by a gradual increase in the concentration of metal ion to determine a saturated extraction capacity. A value of the extraction capacity qₑ (mg/g) was calculated using Equation 3:

$${q}_{e} = \frac{{C}_{in} - {C}_{eq}}{{m}_{E}} \times {V}_{aq} \times {M}_{M} ; \qquad Equation 3$$

where

Cᵢₙ and C_{eq} (mM) represent the concentration of the metal ions in the aqueous phase before extraction and a concentration after reaching equilibrium, respectively, m_{E} is a mass of the extractant used, V_{aq} (L) is a volume of the aqueous phase, and MM is a relative atomic mass of the Au (196.97 g/mol) and the Pd (106.42 g/mol).

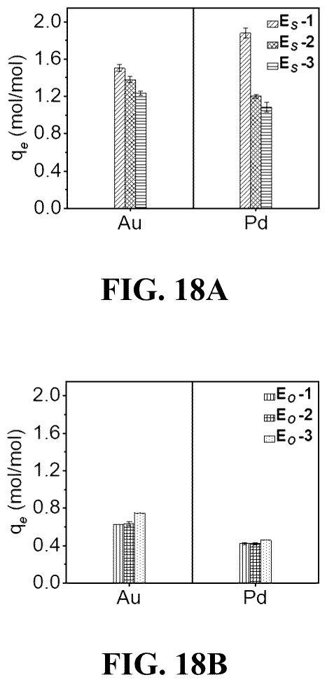

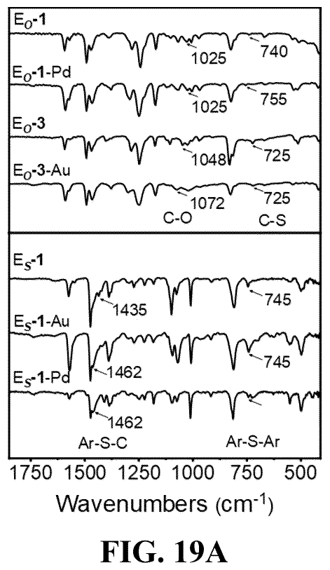

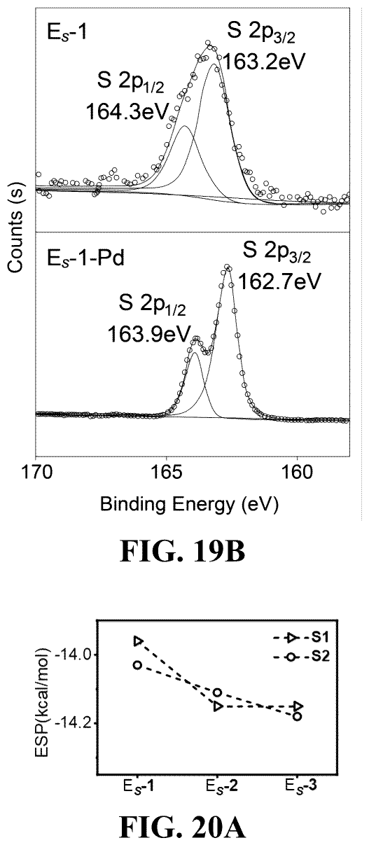

FIG. 18A to FIG. 19B focus on investigating the role of the functional atoms in the extraction capacity. Among these, FIG. 18A shows maximum extraction capacities of E_{S} for the Au(III) and the Pd(II). FIG. 18B shows maximum extraction capacities of E_{O} for the Au(III) and the Pd(II). FIG. 19A shows FT-IR spectra of the extractants in the present disclosure before and after coordination with the Au(III) and the Pd(II). FIG. 19B shows XPS deconvolution results of S·2p spectra for the E_{S}−1 and E_{S}−1−Pd.

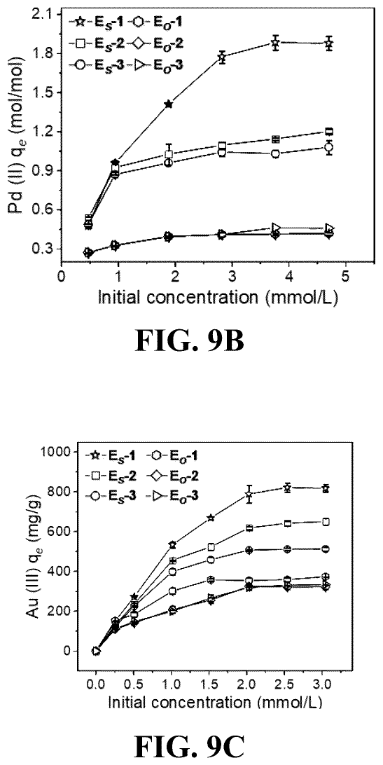

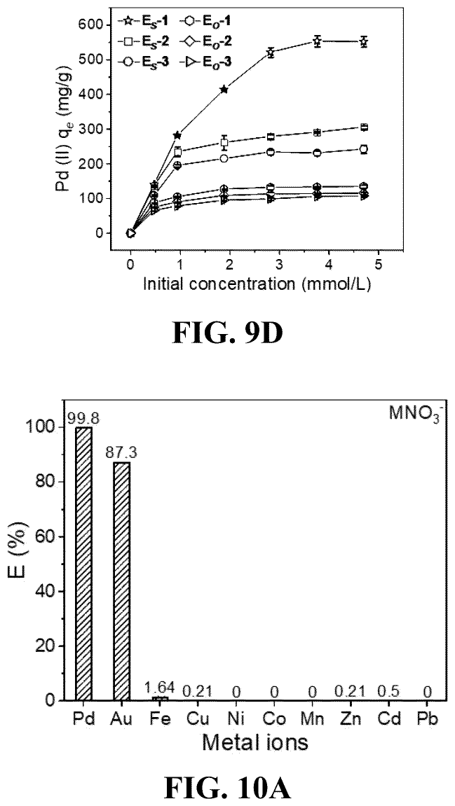

From FIG. 9A to FIG. 9D (FIG. 9A showing the saturated extraction capacities (mol/mol) of the extractants for the Au(III), FIG. 9B showing the saturated extraction capacities (mol/mol) of the extractants for the Pd(II), FIG. 9C showing saturated extraction capacities (mg/g) of the extractants for the Au(III), and FIG. 9D showing saturated extraction capacities (mg/g) of the extractants for the Pd(II) under test conditions: [E_{S} and E_{O}]=1 mM, [Au(III)]=0.25-3 mM, [Pd(II)]=0.5-5 mM, [HCl]=0.1 M), it could be observed that the extraction capacity of the extractants increased with rising a number of the metal ions until reaching equilibrium. Saturated states of these extractants were achieved at concentrations of the Au(III) and the Pd(II) being 2.0 mM and 3.0 mM, respectively. For the extraction of the Au(III) and the Pd(II), the maximum extraction capacities followed the order E_{S}−1>E_{S}−2>E_{S}−3>E_{O}−3>E_{O}−2>E_{O}−1 (see FIGS. 18A-18B). These results indicate the superior performance of the E_{S} over the E_{O} in extraction capacity. As previously noted, the S atom, acting as a stronger soft base compared to the O atom, exhibits easy binding to the Au(III), thereby improving the extraction performance of the Au. In particular, the diphenyl sulfide E_{S}−1 with the shortest carbon chain demonstrates the highest extraction capacity, aligning with the trends observed in FIGS. 16A-16B. Notably, the E_{S}−1 achieved extraction capacities of 818.15 mg/g for the Au(III) and 522.12 mg/g for the Pd(II) (FIG. 9C and FIG. 9D), significantly surpassing most reported extractants, underscoring its potential for efficient recovery of the Au and the Pd from the secondary resources.

From the above results, it was evident that the carbon chain length in diphenyl sulfide compounds results in distinct effects on the extraction behaviors of the E_{S} and the E_{O}. An elongation of carbon chains simultaneously introduces increased steric hindrance and elevated electron cloud density at the functional atoms. While the steric hindrance might suppress metal ion binding, the elevated electron cloud density strengthened interactions between the extractants and the Au(III). Consequently, in the diphenyl sulfide extractants, a type of the functional atoms (S or O) leads to fundamentally opposing structure-selectivity relationships under carbon chain length variations.

The extraction of the metal ions by the extractants primarily relies on binding between the functional atoms and the metal ions. To investigate their specific interactions, Fourier transform infrared (FT-IR) spectroscopy was conducted on the extractants before and after the extraction of the Au(III) and the Pd(II). As shown in FIG. 19A, pristine E_{S}−1 displays two C—S absorption peaks: one at 745 cm⁻¹ corresponding to a Ar—S—Ar linkage formed by two benzene rings, and another at 1,435 cm⁻¹ associated with alkyl chains at both ends of a benzene ring. After the extraction of the Au(III), the C—S peak at 745 cm⁻¹ remains unchanged, while the 1,435 cm⁻¹ peak shifts to 1,462 cm⁻¹, and the peak shape changes obviously. This indicates the binding occurs at two S atoms rather than a central S atom between the benzene rings, likely due to steric hindrance preventing AuCl₄⁻ binding. Similarly, a C—O peak of the E_{O} shifts from 1,048 cm⁻¹ to 1,072 cm⁻¹, while its C—S peak at 725 cm⁻¹ remains stable, which indicates that primary binding sites of the E_{O} in extracting Au(III) are O atoms located on both sides of diphenyl sulfide structure. For the extraction of Pd(II), both C—S peaks in spectrum of E_{S}−1−Au exhibit significant shifts, which indicates that all three S atoms in the E_{S} interacted with the Pd(II). This is corroborated by XPS results (FIG. 19B): S 2p1/2 and S 2p3/2 peaks of the E_{S}−1 at 164.3 eV and 163.2 eV shift to 163.9 eV and 162.7 eV, respectively, after binding with the Pd. In contrast, E_{O} shows a shifted C—S peak (740 cm⁻¹ to 755 cm⁻¹) but unchanged C—O peak (1025 cm⁻¹) post-extraction of the Pd(II). This indicates only the central S atom participates in the extraction of the Pd(II), a phenomenon extensively reported. While the side O atoms of the E_{O} shows negligible influence on Pd coordination, which also explains the slight difference in the minimal capacity among the E_{O}−1, the E_{O}−2 and the E_{O}−3. According to the above results, it can be concluded that the S atoms on both sides of the E_{S} could extract both the Au and the Pd, whereas the central S atom could only extract the Pd. For using E_{O} to extract PM, Au is coordinated by the O atoms on both sides of the E_{O}, while the central S atom could only extract the Pd.

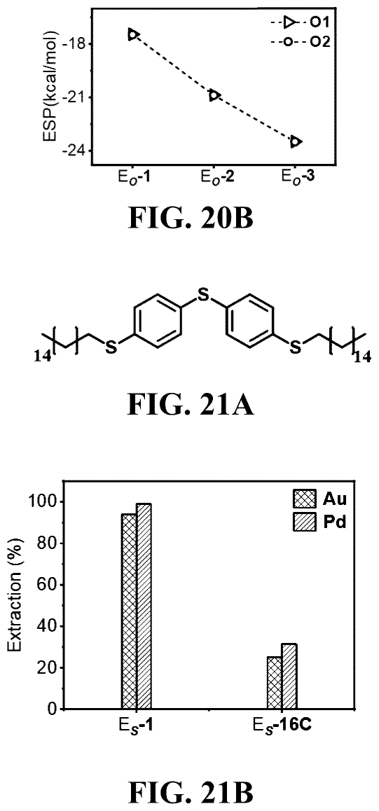

To further investigate the role of the functional atoms in revealing the structure-selectivity relationships via carbon chain length modulation, density functional theory (DFT) calculations were employed to analyze electrostatic potential (ESP) of the extractants. As shown in FIGS. 20A-20B, increasing the carbon chain length leads to ESP of the functional atoms (O or S) on both sides of the diphenyl sulfide structure decreased by 1.1% to 1.4% for the E_{S}, and 34.4% to 34.5% for the E_{O}, respectively. This indicates that elongating terminal carbon chains significantly enhanced the electronegativity of the 0 atoms in the E_{O}, while exerting minimal impact on the S atoms in the E_{S}. As reported previously, atoms with higher electronegativity favored binding of the extractant with the AuCl₄. Consequently, using the AuCl₄⁻ as a model ion, adsorption energies between different extractants and the AuCl₄⁻ were calculated. Table 1 reveals that the E_{O}−3 with the longest carbon chain exhibits the lowest adsorption energy (−131.12 kJ/mol) for [E_{O}][AuCl₄⁻], indicating its superior Au extraction capability over the E_{O}−1 and the E_{O}−2. Surprisingly, despite the relatively higher ESP of the S atoms in the E_{S}−1, its adsorption energy with AuCl₄⁻ was calculated as −148.94 kJ/mol, significantly lower than those of the E_{S}−2 and the E_{S}−3 on AuCl₄ (Table 2). This is contrary to corresponding ESP results, indicating that the ESP of functional atoms is not the main factor to improve the extraction performance of the E_{S}.

TABLE 1
Adsorption energy of E_{O} and AuCl₄⁻

| | | Adsorption | Adsorption |
|---|---|---|---|
| | Energy | energy | energy |
| Structure | (kJ/mol) | (kJ/mol) | (kcal/mol) |
| AuCl₄⁻ | −5190373.134 | / | / |
| E_{O}-1 | −3483770.87 | / | / |
| E_{O}-2 | −3896844.504 | / | / |
| E_{O}-3 | −4308771.291 | / | / |
| [E_{O}-1][AuCl₄⁻] | −8674232.349 | −88.34 | −21.1006 |
| [E_{O}-2][AuCl₄⁻] | −9087331.364 | −113.73 | −27.163 |
| [E_{O}-3][AuCl₄⁻] | −9499275.549 | −131.12 | −31.3185 |

TABLE 2
Adsorption energy of E_{S} and AuCl₄⁻

| | | Adsorption | Adsorption |
|---|---|---|---|
| | Energy | energy | energy |
| Structure | (kJ/mol) | (kJ/mol) | (kcal/mol) |
| AuCl₄⁻ | −5190373.134 | / | / |
| E_{S}-1 | −5179726.39 | / | / |
| E_{S}-2 | −5592799.96 | / | / |
| E_{S}-3 | −6005873.22 | / | / |
| [E_{S}-1][AuCl₄⁻] | −10370248.46 | −148.94 | −35.5734 |
| [E_{S}-2][AuCl₄⁻] | −10783294.25 | −121.16 | −28.9377 |
| [E_{S}-3][AuCl₄⁻] | −11196352.03 | −105.68 | −25.2408 |

It was found that structures with longer side chains exhibit stronger steric hindrance and weaker van der Waals interactions in both E_{S} and E_{O} extractants. To quantitatively assess the impact of steric obstacles and enhanced electrostatic effects (caused by carbon chain elongation) on the attraction of AuCl₄⁻ to surfaces of the E_{S} extractant, this research employed sobEDA. This method enables energy decomposition analysis of adsorption energies between the different extractants and the AuCl₄⁻, thereby providing quantitative evaluation of interaction variation mechanisms from an energetic perspective.

An adsorption energy decomposition equation obtained by the sobEDA is as follows:

$$Δ {E}_{int} = Δ {E}_{els} + Δ {E}_{x} + Δ {E}_{rep} + Δ {E}_{orb} + Δ {E}_{{DFT}_{c}} + Δ {E}_{dc} ; \qquad Equation 4$$

where

ΔEₓ represents exchange interaction energy; ΔE_{DFTc} represents DFT correlation energy, reflecting the contribution of the Coulomb correlation effect to interaction energy between fragments; ΔEᵣₑₚ represents Pauli repulsion energy; ΔE_{orb} represents orbital interaction energy; and ΔE_{dc} represents dispersion correction energy.

FIG. 20A to FIG. 21B shows theoretical calculations for mechanistic investigation. FIG. 20A shows an electrostatic potential (ESP) map of the E_{S}. FIG. 20B shows an electrostatic potential (ESP) map of the E_{O}. FIG. 21A shows structure of E_{S}−16C. FIG. 21B shows comparison of extraction efficiency between the E_{S}−1 and the E_{S}−16C. Test conditions: [E_{S}]=1 mM, [Au(III)]=[Pd(II)]=1 mM, [HCl]=0.1 M, and 4 hours.

Steric hindrance arise from the exchange antisymmetry of electrons, with ΔEₓ and ΔEᵣₑₚ collectively reflecting its magnitude (defined as ΔEₓᵣₑₚ=ΔEₓ+ΔEᵣₑₚ). As the carbon chain lengthened, the ΔEₓᵣₑₚ of the E_{S} progressively increased from 40.38 kcal/mol to 45.07 kcal/mol and 54.78 kcal/mol (Table 3). This increase substantially surpasses variations in ΔEₑₗₛ associated with electrostatic interactions. Based on these results, it was inferred that the electronegativity of the oxygen atoms dominates the extraction of the Au(III) in the E_{O}. For the E_{S}, although the electronegativity of the sulfur atoms improves to some extent with carbon chain elongation, the resulting steric obstacles exert a more pronounced influence on the extraction performance.

TABLE 3
Various sobEDA terms between E_{S} and AuCl₄⁻

| Energy (kcal/mol) | E_{S}-1 | E_{S}-2 | E_{S}-3 |
|---|---|---|---|
| ΔEₑₗₛ | −18.79 | −20.61 | −22.89 |
| ΔEₓ | −14.53 | −15.94 | −18.04 |
| ΔEᵣₑₚ | 54.91 | 61.01 | 72.82 |
| ΔE_{orb} | −10.24 | −12.41 | −12.94 |
| ΔE_{DFTc} | −18.91 | −17.49 | −17.46 |
| ΔE_{dc} | −24.53 | −24.98 | −24.87 |
| ΔEₓᵣₑₚ | 40.38 | 45.07 | 54.78 |

To validate the effect of the carbon chain length on the extraction performance, a side carbon chain of the E_{S} was extended to 16 carbons (designated as E_{S}−16C) (FIG. 21A), and recovery tests for the Au and the Pd were conducted (FIG. 21B). Under identical testing conditions, the extraction efficiencies for the Au(III) and the Pd(II) decreased by 73.3% and 68.2%, respectively, further confirming the significant influence of the steric hindrance on extraction efficiency.

### Study on the Extraction Mechanism of the Au(III) and the Pd(II)

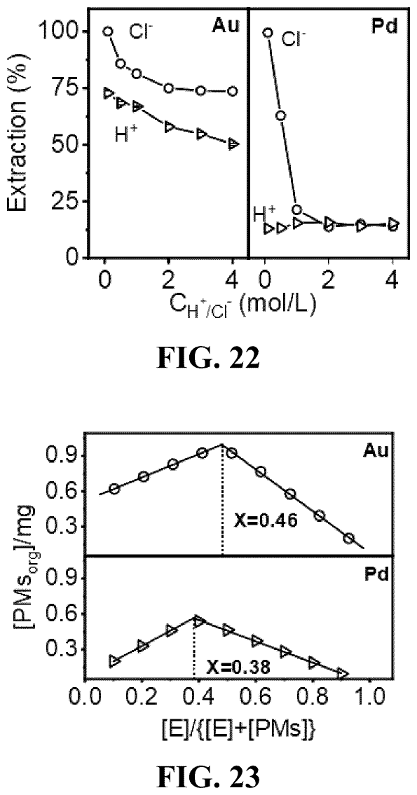

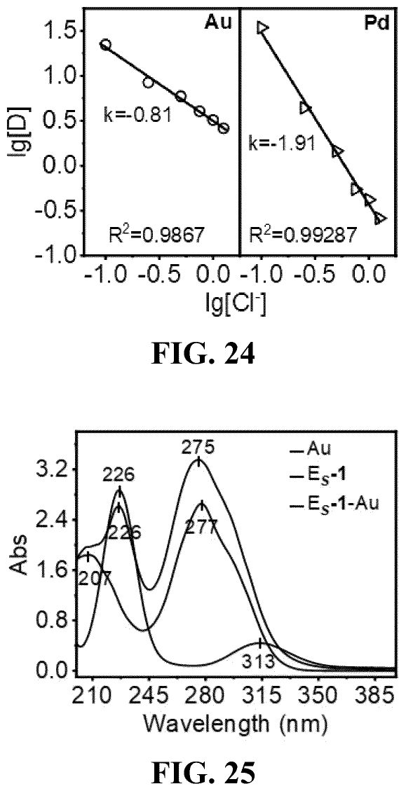

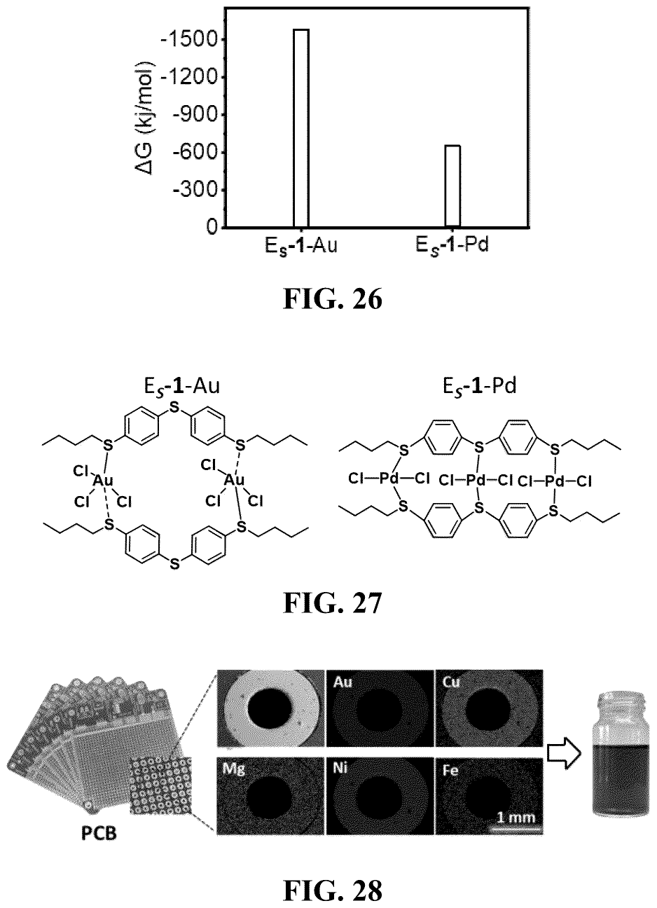

FIG. 22 to FIG. 27 shows the extraction mechanisms. FIG. 22 shows influences of H⁺ and Cl⁻ concentrations on the extraction performance of the Au(III) and the Pd(II). Under testing conditions: [E_{S}−1]=2 mM, [Au(III)]=[Pd(II)]=1 mM, FIG. 23 shows Job's plots for E_{S}−1−Au(III) and E_{S}−1−Pd(II) complexes; and FIG. 24 shows relationship between log [D] and log [C₁]. Under testing conditions ([E_{S}−1]=2 mM, [Au(III)]=[Pd(II)]=1 mM), FIG. 25 shows UV-Vis spectra of the E_{S} before and after the extraction of the Au(III); and FIG. 26 shows dual descriptor of the E_{S}−1. FIG. 27 shows optimized structures of E_{S}−1−Au and E_{S}−1−Pd complexes. The extraction methodology for FIG. 22 to FIG. 27 followed these steps: the extractant was weighed and dissolved in DCM to form an organic phase; an aqueous phase consisted of HCl and PM ions (Au(III) or Pd(II)); subsequently, a mixture of the organic phase and the aqueous phase was mechanically oscillated at 230 rpm under 25° C., with compositions adjusted according to specific test conditions.

Note that when the present disclosure studies the impact of different test conditions on test results, test methods were the same as described above, and could be set accordingly based on the different parameters to be studied.

The mechanism of extracting the Au(III) and the Pd(II) by the E_{S}−1 was investigated in this study. Since extraction of metal occurs in a hydrochloric acid medium, the influences of the H⁺ and Cl⁻ concentrations on the extraction performance were first examined. To vary the Cl-concentration (0.1 M to 4 M), a certain amount of NaCl was added to a 0.1 M HCl solution. Similarly, in order to control the H⁺ concentration (0.1 M to 4 M), hydrochloric acid was supplemented while maintaining the Cl⁻ concentration at 4 M through additional NaCl. As shown in FIG. 22, increased concentrations of both H⁺ and Cl⁻ reduce the extraction performance for Au(III). On one hand, elevated H⁺ levels inhibited HAuCl₄⁻ hydrolysis, thereby weakening AuCl₄ binding with the extractant. On the other hand, the generation of Cl⁻ ions during the extraction likely hindered the extraction reaction. For the extraction of the Pd(II), H⁺ concentration variations show negligible impact on H₂PdCl₄ hydrolysis. Only increased Cl⁻ concentration adversely affects the extraction performance for Pd(II), indicating Cl⁻ generation during the extraction.

To further investigate the binding between the extractant and the PM ions, stoichiometric ratios were analyzed using Job's method. Briefly, a total volume was maintained at 10 mL, and a contact volume ratio of the organic phase to the aqueous phase was adjusted from 1:9 to 9:1, while ensuring a concentration of the E_{S}−1 and the metal ions remained at 1 mM. The concentrations of the metal ions in both the organic phase and the aqueous phase were measured and calculated based on the principle of mass conservation. Linear regression analysis was subsequently conducted to determine coordination stoichiometry. As shown in FIG. 23, the concentrations of the Au(III) and the Pd(II) in the organic phase peaked when the mole fractions of E_{S}−1 reached 0.47 and 0.38, respectively, indicating that ratios of the complexes formed by the E_{S}−1 with the Au(III) and the Pd(II) were 1:1.1 and 1:1.6, respectively. This corresponded to the binding of one Au(III) ion per E_{S}−1 molecule, while three Pd(II) ions are bound by two E_{S}−1 molecules.

To determine state of the Au(III) and the Pd(II) in the form of complex after extraction, stoichiometric ratios of the complexes were determined by plotting relationship between log [D](distribution ratio) and log [Cl⁻]. Specifically, the concentrations of the H⁺, the PM ions, and the extractant were fixed at 0.1 M, 1 mM, and 2 mM, respectively. The concentration of Cl⁻ was varied by adding the NaCl to investigate its influence on log [D] during the extraction of the Au(III) and the Pd(II) using the E_{S}−1. After plotting the relationship between the log [D] and the log [Cl⁻], a absolute value of the slope (k) of an obtained straight line was interpreted as a stoichiometric coefficient of the Cl⁻ in a chemical equation. As shown in FIG. 24, the slope for the extraction of the Au(III) is −0.81, indicating the generation of one Cl⁻ ion per AuCl₄⁻ involved in the reaction. This is further confirmed by UV-Vis characterization (FIG. 25), where an absorption peak of the Au(III) at 313 nm disappeared after binding with the E_{S}−1, suggesting a change in state of the Au during the extraction. For the extraction of the Pd(II), a slope of a fitting line is −1.91, implying release of two Cl⁻ ions. These results demonstrate that the extraction of the Au(III) and the Pd(II) by the E_{S} proceeds via a coordination substitution mechanism.

Combining the results of the Job's method, chemometric studies, and various characterizations, the extraction mechanism of the Au(III) and the Pd(II) with E_{S} is described as follows:

$$E + {AuCl}_{4}^{-} ⇌ [ E \cdot {AuCl}_{3} ] + {Cl}^{-} ; \qquad Equation 5$$

$$2 E + 3 {PdCl}_{4}^{2 -} ⇌ [ 2 E \cdot 3 {PdCl}_{2} ] + 6 {Cl}^{-} ; \qquad Equation 6$$

Gibbs free energy changes (ΔG) calculated for the extraction of the Au(III) and the Pd(II) processes were −1,581.69 kJ/mol and −654.93 kJ/mol, respectively (FIG. 26 and Table 4). This indicates that reactions were thermodynamically spontaneous and supporting the feasibility of the proposed extraction mechanism. To better understand complex structures and further elucidate the extraction mechanism, active sites of the E_{S}−1 were confirmed using dual descriptor. In the E_{S}−1, an area near the sulfur atom and parallel to a benzene ring plane was identified as a site for electrophilic interactions with nucleophilic ions such as AuCl₄⁻ and PdCl₄²⁻. Based on a derived mechanism and the dual descriptor of the E_{S}−1, the structures of the complexes are shown in FIG. 27. The paired structure of the extractant highlights the superior extraction performance of the E_{S}, underscoring its significant potential for recovering the PMs from the secondary resources.

TABLE 4
Binding energy of E_{S}-1 and PMs

| Structure | Energy/kJ/mol | Binding energy/kJ/mol |
|---|---|---|
| E_{S}-1 | −5179809.76 | / |
| Cl⁻ | −1208809.28 | / |
| AuCl₄⁻ | −5189884.51 | / |
| E_{S}-1-Au | −18323351.68 | −1581.69 |
| PdCl₄²⁻ | −5170505.854 | / |
| E_{S}-1-Pd | −18618936.34 | −654.93 |

Selective Recovery of the Au(III) and the Pd(II) from the Secondary Resources

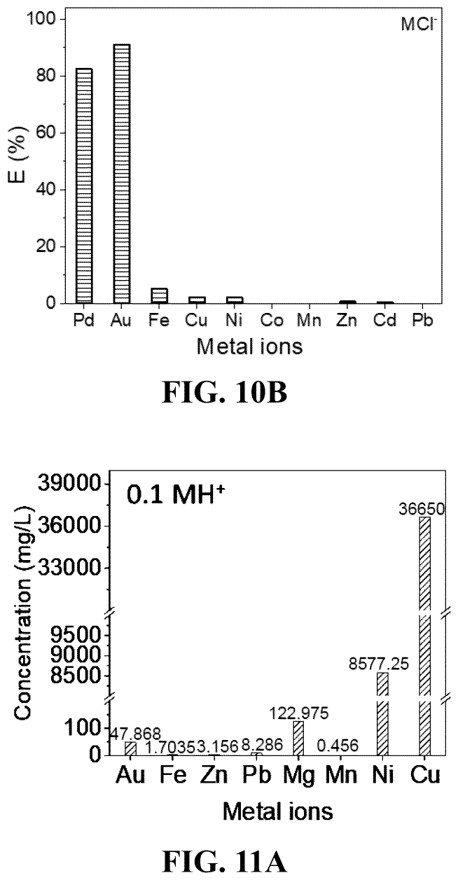

FIG. 10A shows the extraction of the Au(III) and the Pd(II) from simulated mixed metal ion solutions; FIG. 10 shows the extraction of the Au(III) and the Pd(II) from simulated mixed metal ion solutions; test conditions: [E_{S}−1]=2 mM, [metal ion]=1 mM, [HCl]=0.1 M, O/A=1, 25° C., 4 hours.

The experimental procedure was conducted as follows: a 50 mL solution with a concentration of metal ions being 1 mM was prepared by weighing required amounts (50 mL) of respective metal salts and diluting to 50 mL using a volumetric flask. The extraction experiments followed the same protocol as described earlier. An organic phase consisted of E_{S}−1 dissolved in DCM, while a aqueous phase was a mixed-metal solution prepared above. Equal volumes (5 mL each) of the organic phase and the aqueous phase were mixed and mechanically oscillated.

Selective extraction of the PM ions from complex polymetallic solutions is crucial for efficient resource recovery. In this study, a simulated mixed-metal solution containing 1 mM Pd(II), Au(III), and nitrate or chloride salts of Fe(III), Cu(II), Ni(II), Co(II), Mn(II), Zn(II), Cd(II), and Pb(II) was prepared to verify the selectivity of the E_{S}. As shown in FIG. 10A, the E_{S}−1 achieves extraction efficiencies of 87.3% for the Au(III) and 99.8% for the Pd(II) in nitrate-based systems, while extraction of base metals remains below 2%. In chloride-based systems, extraction efficiencies for the Au(III) and the Pd(II) reaches 91.11% and 82.52%, respectively. Notably, the lower extraction efficiency of the Pd(II) in the chloride-based systems is attributed to interference from Cl⁻, as detailed in FIG. 10B. These results underscore remarkable potential of the E_{S} for selectively extracting the Au(III) and the Pd(II) in complex polymetallic systems.

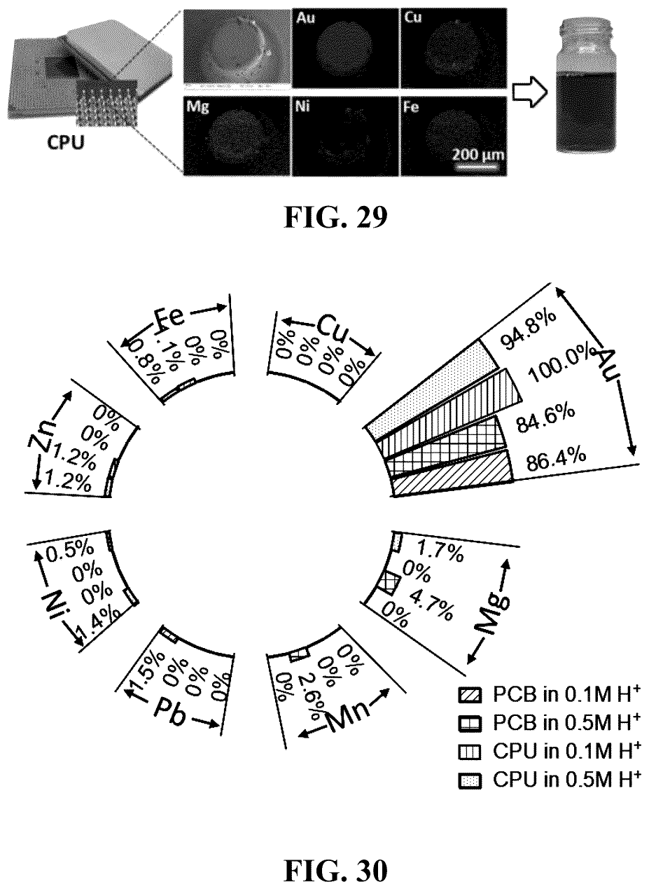

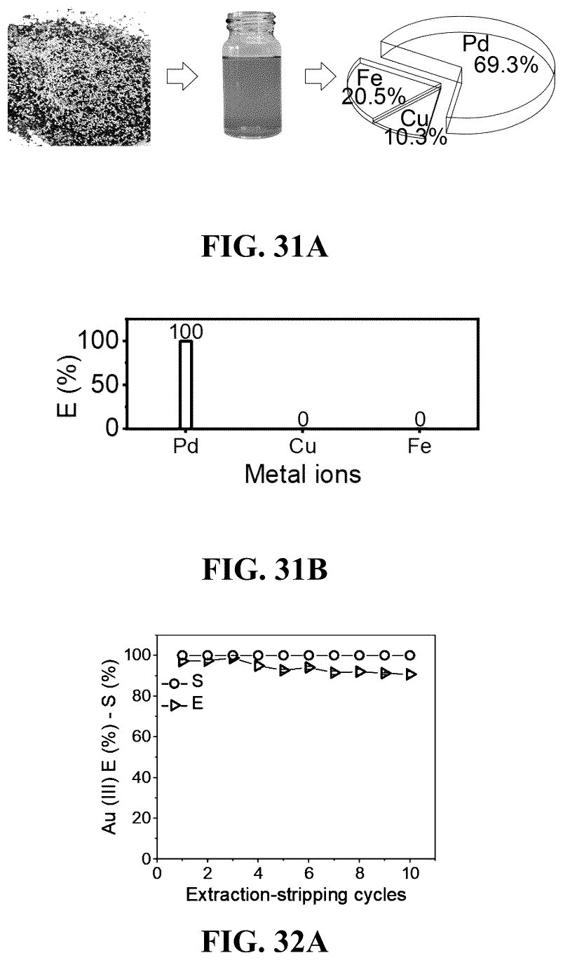

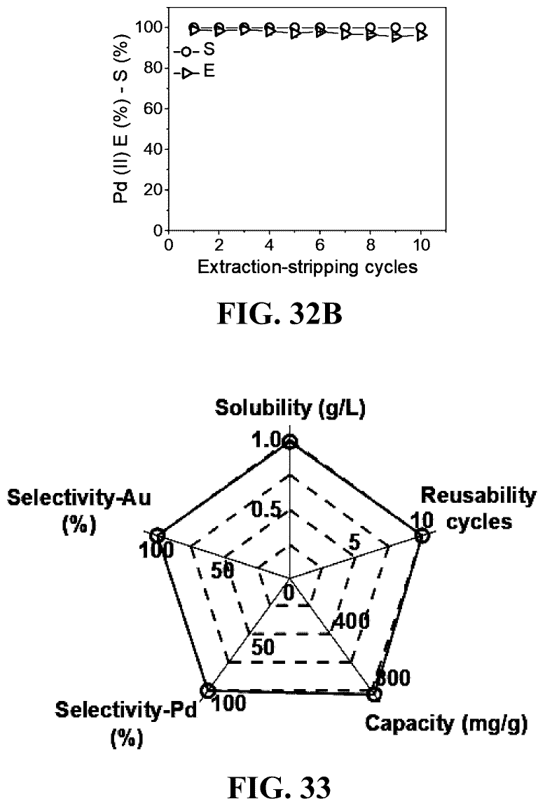

FIG. 28 to FIG. 33 show the performance of recovering the Au(III) and the Pd(II) from the secondary resources, including SEM images, elemental mapping, and corresponding leachates. FIG. 28 shows SEM images of real-world PCB boards and their leachate solutions. FIG. 29 shows SEM images of real-world CPUs and their leaching solutions. FIG. 30 shows Au recovered using 2 mM E_{S}−1 under H⁺ concentrations of 0.1 M and 0.5 M. FIG. 31A shows initial ion concentrations in a spent catalyst leachate dissolved by HNO₃. FIG. 31B shows the extraction performance of the E_{S}−1 for the spent catalyst leachate. FIG. 32A shows cyclic extraction-stripping tests for the Au. FIG. 32B shows cyclic extraction-stripping tests for the Pd. Test conditions: [E_{S}−1]=2 mM, [Au(III)]=[Pd(II)]=1 mM, [HCl]=0.1 M, using 1 M thiourea in 0.5 M HCl for elution at 25° C. for 4 hours. FIG. 33 shows a schematic overview for the comprehensive performance of the E_{S}−1. The specific testing methods followed those described in Use Examples 1 to 3.

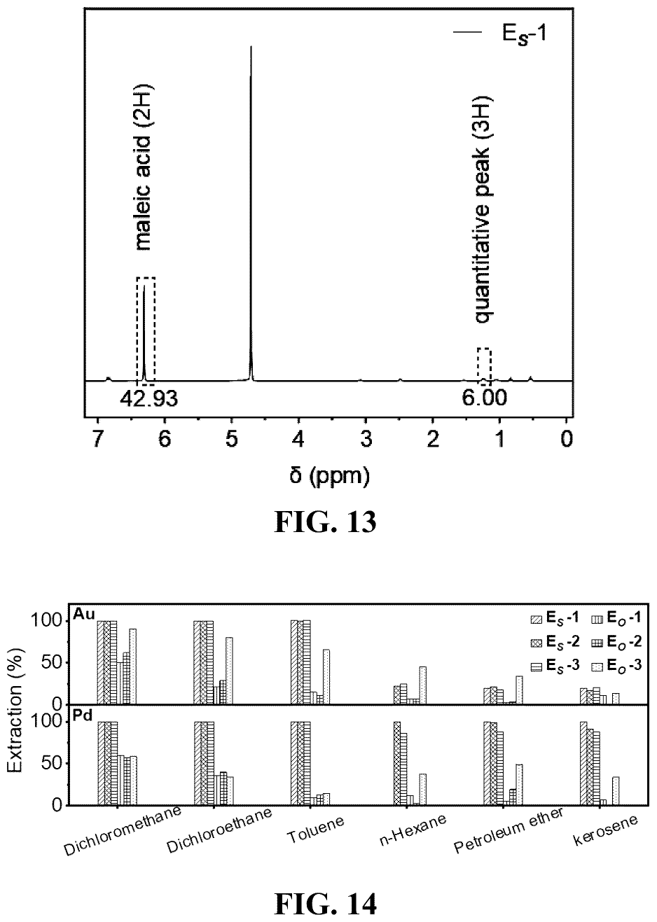

Subsequently, the feasibility of the E_{S}−1 was evaluated for recovering the PMs from real-world PCB, CPU, and waste Pd catalyst leachates. Here, PCB and CPU boards were selected as representative Au-containing electronic waste (FIG. 28 to FIG. 29). In leachates with varying H concentrations, the E_{S} achieved near-complete Au(III) recovery (extraction efficiency: 94.8% to 100%) at a minimal concentration of 2 mM without extracting coexisting metal ions (FIG. 30, with percentages indicating extraction efficiencies). Notably, the E_{S} failed to extract Cu, the predominant component in a CPU leachate, across various media and concentrations, even at an exceptionally high concentration of 1.87×10⁵ mg/L (see FIGS. 11A-11D, where FIG. 11A shows metal concentrations in the PCB leachate, FIG. 11B shows metal concentrations in the PCB leachate, FIG. 11C shows metal concentrations in the CPU leachate, and FIG. 11D shows metal concentrations in the CPU leachate). Additionally, the waste Pd catalysts containing Pd, Fe, and Cu ions were chosen as Pd-based secondary resources for practical Pd recovery (FIG. 31A). It is exciting that the E_{S}−1 achieved effective and precise Pd recovery (extraction efficiency >99.99%) without co-extracting coexisting metal ions (FIG. 31). These results highlight the practical viability and exceptional performance of the E_{S} in the PM recovery, primarily attributed to its coordination mechanism and stable complexation maintained in its paired structure. Furthermore, the E_{S}−1 enables repeated use through established stripping methods using stripping agents (e.g., thiourea and HCl) to recover the PMs from E_{S}−1−Au and E_{S}−1−Pd complexes. As shown in FIGS. 32A-32B, the extraction efficiency of the E_{S}−1 decreases by 6.5% for the Au and approximately 2.6% for the Pd with increasing extraction-stripping cycles. Even after ten cycles, its extraction performance for both Au and Pd remains high, confirming excellent reusability. FT-IR analysis on the E_{S}−1 after multiple elution cycles reveals well-preserved characteristic functional group peaks (see FIG. 12, FIG. 12 showing FT-IR spectra of the E_{S}−1 after stripping of the Au(III) and the Pd(II)). The water solubility of the extractant, a critical physicochemical property for assessing hydrophilicity and operational stability of the extractant, was further evaluated through quantitative NMR (q-NMR). As shown in FIG. 13 (FIG. 13 showing a q-NMR spectrum for the solubility of the E_{S}−1 in water) and Table 5, the E_{S}−1 exhibited a calculated solubility of 0.987 g/L in water, indicating favorable aqueous stability. Combined with its high capacity, exceptional selectivity, low solubility, and remarkable reusability (see FIG. 33), the E_{S}−1 emerges as a compelling candidate for efficient PM recovery from the secondary resources.

TABLE 5
q-NMR parameters of E_{S}-1

| | | A_{Ext} | A_{Maleic acid} | N_{Maleic acid} | NExt | |
|---|---|---|---|---|---|---|
| | | (extractant, H- | (maleic acid, | (maleic acid, | (E_{S}-1, | |
| | V_{D2O} | NMR peak | H-NMR | molar | molar | Solubility |
| Extractant | (mL) | area | peak area) | amount) | amount) | (g/L) |
| E_{S}-1 | 0.8 | 6 | 42.93 | 0.0258 | 0.003606 | 0.987 |

Although the present disclosure is described in detail in conjunction with the foregoing examples, they are only a part of, not all of, the embodiments of the present disclosure. Other examples could be obtained based on these embodiments without creative efforts, and all of these embodiments shall fall within the scope of the present disclosure.

What is claimed is:

1. A structurally symmetrical diphenyl sulfide compound, having a structure selected from the group consisting of Formula I and Formula II: 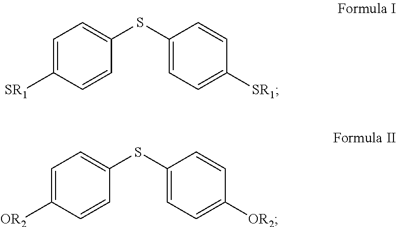
wherein R₁ in the Formula I is a C4-C8 alkyl group; and R₂ in the Formula II is a C4-C8 alkyl group.

2. The structurally symmetrical diphenyl sulfide compound of claim 1, wherein R₁ and R₂ each are independently selected from the group consisting of —C₄H₉, —C₆H₁₃, and —C₈H₁₇.

3. A method for preparing the structurally symmetrical diphenyl sulfide compound of claim 1, comprising the following steps:
mixing mercapto diphenyl sulfide or hydroxy diphenyl sulfide, a halogenated alkane, an organic solvent, an acid-binding agent, and an organic ammonium salt catalyst, and subjecting a resulting mixture to substitution to obtain the diphenyl sulfide compound,
wherein an alkane in the halogenated alkane is a C4-C8 alkane; the mercapto diphenyl sulfide comprises 4,4′-dimercapto diphenyl sulfide; and the hydroxy diphenyl sulfide comprises 4,4′-dihydroxy diphenyl sulfide.

4. The method of claim 3, wherein R₁ and R₂ each are independently selected from the group consisting of —C₄H₉, —C₆H₁₃, and —C₈H₁₇.

5. The method of claim 3, wherein the halogenated alkane comprises a halogenated group selected from the group consisting of a halogenated butyl group, a halogenated hexyl group, and a halogenated octyl group.

6. The method of claim 3, wherein the substitution is conducted at a temperature of 70° C. to 90° C. for 24 hours to 60 hours.

7. A use method of the structurally symmetrical diphenyl sulfide compound of claim 1, comprising using the diphenyl sulfide compound as an extractant in extraction of at least one selected from the group consisting of Au³⁺ and Pd²⁺.

8. The use method of claim 7, wherein the extraction comprises:
mixing an acidic leachate containing at least one selected from the group consisting of the Au³⁺ and the Pd²⁺ with an extractant dilution solution, and subjecting a resulting mixed solution to the extraction.

9. The use method of claim 7, wherein hydrogen ions in the acidic leachate have a concentration of 0.1 mol/L to 18 mol/L.

10. The use method of claim 7, wherein a diluent in the extractant dilution solution comprises at least one selected from the group consisting of dichloromethane (DCM), dichloroethane (DCE), toluene, n-hexane, petroleum ether, and kerosene; and
the extractant in the extractant dilution solution has a concentration of 1 mmol/L to 10 mmol/L.

11. The use method of claim 7, wherein a concentration of the Au³⁺ in the acidic leachate ranges from 0 mg/L to 80 mg/L, and a concentration of the Pd²⁺ in the acidic leachate ranges from 0 mg/L to 15 mg/L, and the concentration of the Au³⁺ and the concentration of the Pd²⁺ are not 0 simultaneously.
